(19) United States
(12) Patent Application Publication
(10) Pub. No.: US 2015/0282768 A1
(43) Pub. Date: Oct. 8, 2015

(54) Physiological signal determination of bioimpedance signals

(71) Applicants: Michael Edward Smith Luna, San Jose, CA (US); Sidney Primas, Mountain View, CA (US); John Stivoric, San Francisco, CA (US)

(72) Inventors: Michael Edward Smith Luna, San Jose, CA (US); Sidney Primas, Mountain View, CA (US); John Stivoric, San Francisco, CA (US)

(73) Assignee: AliphCom, San Francisco, CA (US)

(21) Appl. No.: 14/121,943

(22) Filed: Nov. 4, 2014

Related U.S. Application Data
(63) Continuation-in-part of application No. 13/831,260, filed on Mar. 14, 2013.
Application No. 13/831,260 is a continuation-in-part of application No. 13/802,305, filed on Mar. 13, 2013.
Application No. 13/802,305 is a continuation-in-part of application No. 13/802,319, filed on Mar. 13, 2013.
Application No. 13/802,319 is a continuation-in-part of application No. 14/480,628, filed on Sep. 8, 2014.

(30) Foreign Application Priority Data
Sep. 29, 2012 (CN) 201220513278.5

(51) Int. Cl. A61B 5/00; A61B 5/0205; A61B 5/053

(52) U.S. Cl. CPC A61B 5/7278; A61B 5/7225; A61B 5/053; A61B 5/0205; A61B 5/681; A61B 5/0533; A61B 5/024

(57) ABSTRACT

Embodiments relate generally to wearable computing devices in capturing and deriving physiological characteristic data. More specifically, disclosed are one or more electrodes and methods to determine physiological characteristics using a wearable device (or carried device) and one or more sensors. In one embodiment, a method includes determining a drive signal magnitude for a bioimpedance signal to capture data representing a physiological-related component and selecting the drive signal magnitude as a function of an impedance of a tissue. The bioimpedance signal can be applied to electrodes that are configured to convey the bioimpedance signal to the tissue. In some cases, data representing a value a signal-to-noise (“SNR”) ratio may be adapted to form an adaptive signal-to-noise value. A portion of a received bioimpedance signal may be detected, the received bioimpedance signal being based on the adaptive signal-to-noise value. A physiological characteristic can be derived.

### CROSS-RELATED APPLICATIONS

This application is a continuation-in-part application of U.S. patent application Ser. No. 13/831,260 (ALI-147) filed on Mar. 14, 2013, which claims priority to Chinese Utility Model Patent Application Number 201220513278.5 filed on Sep. 29, 2012; THIS APPLICATION is a continuation-in-part application of U.S. patent application Ser. No. 13/802,305 (ALI-267) filed on Mar. 13, 2013, which is a continuation-in-part application of U.S. patent application Ser. No. 13/831,260 (ALI-147) filed on Mar. 14, 2013, which claims priority to Chinese Utility Model Patent Application Number 201220513278.5 filed on Sep. 29, 2012; THIS APPLICATION is a continuation-in-part application of U.S. patent application Ser. No. 13/802,319 (ALI-268) filed on Mar. 13, 2013, which is a continuation-in-part application of U.S. patent application Ser. No. 13/831,260 (ALI-147) filed on Mar. 14, 2013, which claims priority to Chinese Utility Model Patent Application Number 201220513278.5 filed on Sep. 29, 2012; THIS APPLICATION is a continuation-in-part application of U.S. patent application Ser. No. 14/480,628 (ALI-516) filed on Sep. 8, 2014, all of which are incorporated by reference herein for all purposes.

### FIELD

Embodiments of the invention relate generally to electrical and electronic hardware, computer software, wired and wireless network communications, and wearable computing devices for facilitating health and wellness-related information. More specifically, disclosed are electrodes and methods to determine physiological characteristics using a wearable device (or carried device) and one or more sensors that can be subject to motion.

### BACKGROUND

Devices and techniques to gather physiological information, such as a heart rate of a person, while often readily available, are not well-suited to capture such information other than by using conventional data capture devices. Conventional devices typically lack capabilities to capture, analyze, communicate, or use physiological-related data in a contextually-meaningful, comprehensive, and efficient manner, such as during the day-to-day activities of a user, including high impact and strenuous exercising or participation in sports. Further, traditional devices and solutions to obtaining physiological information generally require that the sensors remain firmly affixed to the person, such as being affixed to the skin. In some conventional approaches, a few sensors are placed directly on the skin of a person while the sensors and the person are relatively stationary during the measurement process. While functional, the traditional devices and solutions to collecting physiological information are not well-suited for active participants in sports or over the course of over a period of time, such as one or more days.

Conventional biometric sensing devices and techniques to obtaining physiological information are susceptible to motion artifacts in the sensing signals. Typically, motion-related noise typically gives rise to motion artifacts, which usually affect sensing signals generated by sensors. Motion-related noise typically occludes or otherwise distorts sensed physiological signals, such as heart rate, respiration and the like. One example of motion-related noise is electrical noise generated by intermittent contact between sensors and the tissue from which physiological signals are sensed. Another example of motion-related noise is the electrical noise signals generated by nerve firings due in the muscles during contraction and during movement of a person's body. Such electrical noise signals can emanate from electrical impulses of muscles (e.g., as evidenced, in some cases, by electromyography (“EMG”), which is typically used to determine the existence and/or amounts of motion based on electrical signals generated by muscle cells at rest or in contraction).

To reduce or minimize the effects of motion-related noise, traditional approaches generally require a person to remain substantially motionless and/or locate the sensing mechanisms (i.e., sensors) on proximal portions of a person's appendage or limb proximal (i.e., near the point of attachment to a torso of the person, such as at or on the upper arm between the elbow and shoulder). Proximal portions of an appendage or limb generally experience less motion and/or acceleration (or less degrees of motion and/or acceleration) than distal portions of an appendage or limb. Examples of distal portions of appendages or limbs include wrists, ankles, toes, fingers, and the like. Distal portions or locations are those that are furthest away from, for example, a torso relative to the proximal portions or locations. Therefore, conventional biometric sensing devices and techniques, especially those susceptible to motion, are generally located at the proximal portions to reduce or minimize the effects of motion.

When motion is present, traditional biometric sensing devices and techniques are not well-suited to obtain physiological information. Another drawback to traditional biometric sensing devices and techniques is the requirement to locate such devices at proximal portions of a limb. In some cases, the extremities of a person's body typically exhibit the presence of an infirmity, ailment or condition more readily than a person's core (i.e., torso). Thus, sensors co-located at proximal portions of a limb may be less likely to sense or otherwise detect the infirmity, ailment or condition, thereby foregoing opportunities to alert the wearer of physiological changes that may indicate the onset of, for example, sleep or tremors.

Further, co-locating sensors at proximal portions of a limb hinders an ability to determine or predict the onset of a physiological state or a change from one physiological state to another. For example, in some conventional sensing techniques, the detection of the onset of sleep, as well as and the various sleep stages, is typically performed by using sensors located at the proximal regions. By co-locating the sensors at the proximal regions rather than at the extremities of a limb, the prediction of sleep or any other physiological state is made more difficult. As an example, consider the detection of an ailment or malady, such as a diabetic tremor, Parkinson's tremors, and/or an epileptic tremor. The use of sensors at proximal portions of a limb is typically sub-optimal for the detection of such tremors prior to the afflicted person's awareness of such a change in physiological state.

Thus, what is needed is a solution for data capture devices, such as for wearable devices, without the limitations of conventional techniques.

### BRIEF DESCRIPTION OF THE DRAWINGS

Various embodiments or examples (“examples”) of the invention are disclosed in the following detailed description and the accompanying drawings:

FIG. 1A illustrates an exemplary array of electrodes and a physiological information generator disposed in a wearable data-capable band, according to some embodiments;

FIGS. 1B to 1D illustrate examples of electrode arrays, according to some embodiments;

FIG. 2 is a functional diagram depicting a physiological information generator implemented in a wearable device, according to some embodiments;

FIGS. 3A to 3C are cross-sectional views depicting arrays of electrodes including subsets of electrodes adjacent an arm of a wearer, according to some embodiments;

FIG. 4 depicts a portion of an array of electrodes disposed within a housing material of a wearable device, according to some embodiments;

FIG. 5 depicts an example of a physiological information generator, according to some embodiments;

FIG. 6 is an example flow diagram for selecting a sensor, according to some embodiments;

FIG. 7 is an example flow diagram for determining physiological characteristics using a wearable device with arrayed electrodes, according to some embodiments;

FIG. 8 illustrates an exemplary computing platform disposed in a wearable device in accordance with various embodiments

FIG. 9 depicts the physiological signal extractor, according to some embodiments;

FIG. 10 is a flowchart for extracting a physiological signal, according to some embodiments;

FIG. 11 is a block diagram depicting an example of a physiological signal extractor, according to some embodiments;

FIG. 12 depicts an example of an offset generator, according to some embodiments;

FIG. 13 is a flowchart depicting example of a flow for decomposing a sensor signal to form separate signals, according to some embodiments;

FIGS. 14A to 14D depict various signals used for physiological characteristic signal extraction, according to various embodiments;

FIG. 15 depicts recovered signals, according to some embodiments;

FIG. 16 depicts an extracted physiological signal, according to various embodiments;

FIG. 17 illustrates an exemplary computing platform disposed in a wearable device in accordance with various embodiments;

FIG. 18 is a diagram depicting a physiological state determinator configured to receive sensor data originating, for example, at a distal portion of a limb, according to some embodiments;

FIG. 19 depicts a sleep manager, according to some embodiments;

FIG. 20A depicts a wearable device including a skin surface microphone (“SSM”), according to some embodiments;

FIG. 20B depicts an example of data arrangements for physiological characteristics and parametric values that can identify a sleep state, according to some embodiments;

FIG. 21 depicts an anomalous state manager, according to some embodiments;

FIG. 22 depicts an affective state manager configured to receive sensor data derived from bioimpedance signals, according to some embodiments;

FIG. 23 illustrates an exemplary computing platform disposed in a wearable device in accordance with various embodiments;

FIG. 24 is a diagram depicting examples of various components configured to determine one or more physiological characteristics, according to some examples;

FIG. 25 is a diagram of an example of an electrode contact state evaluator, according to some examples;

FIG. 26 is an example of a number of current profiles with which a drive signal adjuster may adjust current, according to some examples;

FIG. 27 is a diagram depicting a signal receiver, according to some examples;

FIG. 28 is a diagram depicting a physiological signal component correlator, according to some examples;

FIG. 29 is a diagram depicting an example of components of a physiological signal component correlator, according to some embodiments;

FIG. 30 is a diagram depicting exemplary operation of a parameter updater, peak variability validator, and an adapted signal-to-noise ratio characterizer, according to various examples; and

FIG. 31 illustrates an exemplary computing platform disposed in a device configured to facilitate physiological signal determination in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

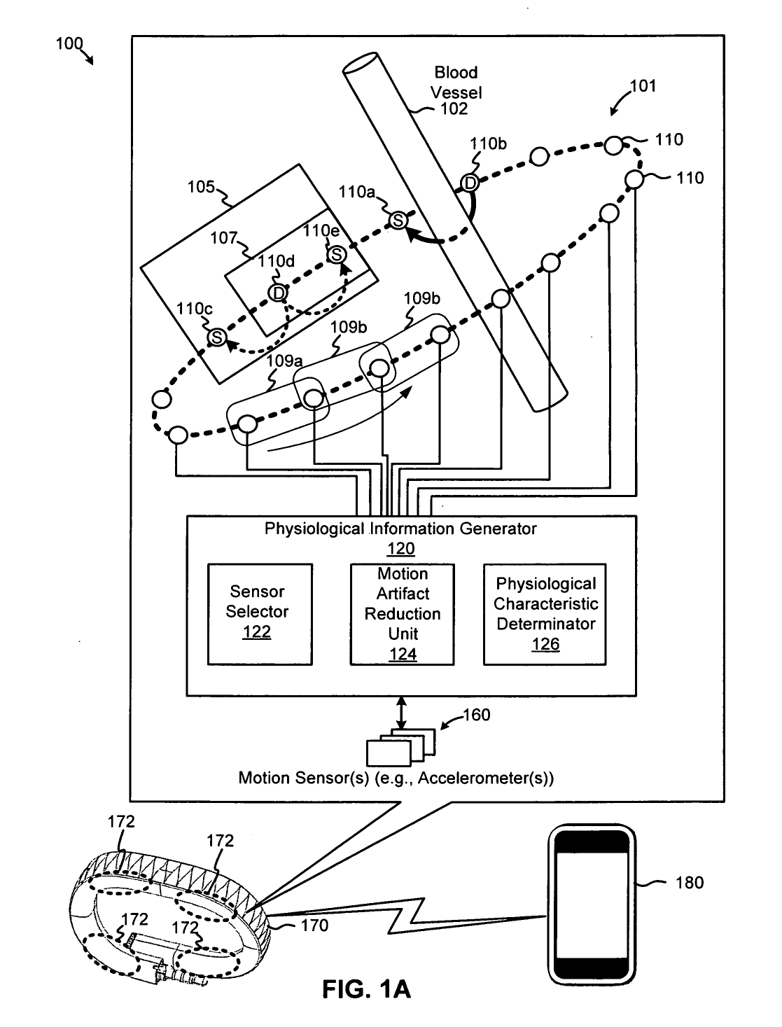

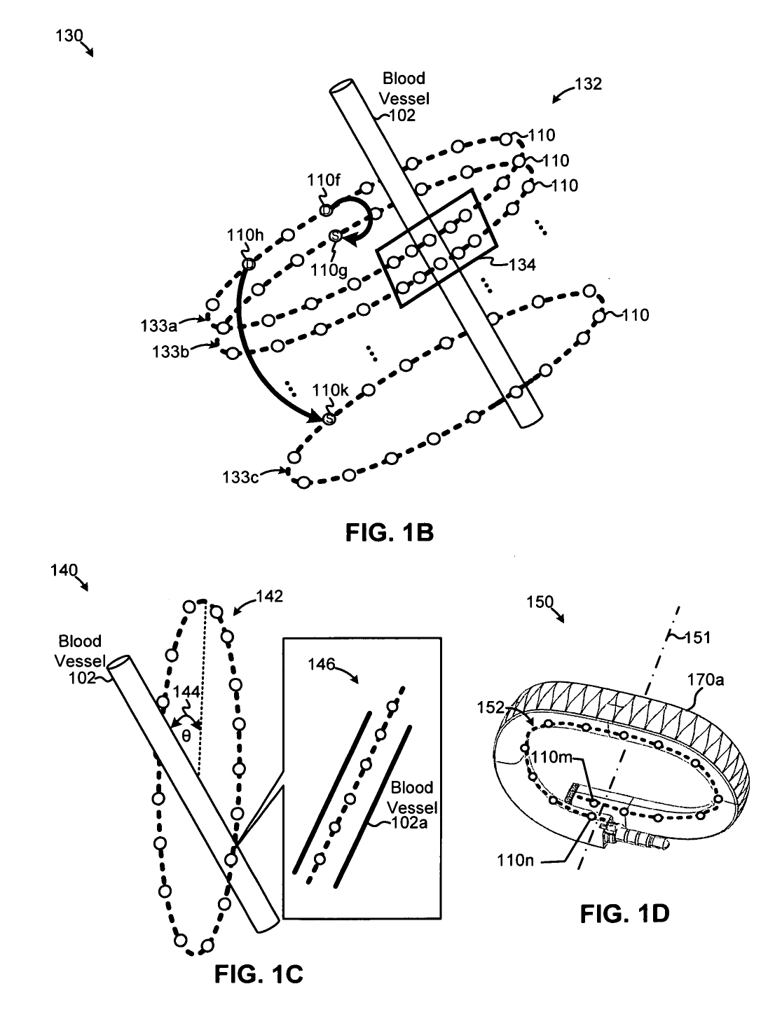

FIG. 1A illustrates an exemplary array of electrodes and a physiological information generator disposed in a wearable data-capable band, according to some embodiments. Diagram 100 depicts an array 100 of electrodes 110 coupled to a physiological information generator 120 that is configured to generate data representing one or more physiological characteristics associated with a user that is wearing or carrying array 101. Also shown are motion sensors 160, which, for example, can include accelerometers. Motion sensors 160 are not limited to accelerometers. Examples of motion sensors 160 can also include gyroscopic sensors, optical motion sensors (e.g., laser or LED motion detectors, such as used in optical mice), magnet-based motion sensors (e.g., detecting magnetic fields, or changes thereof, to detect motion), electromagnetic-based sensors, etc., as well as any sensor configured to detect or determine motion, such as motion sensors based on physiological characteristics (e.g., using electromyography (“EMG”) to determine existence and/or amounts of motion based on electrical signals generated by muscle cells), and the like. Electrodes 110 can include any suitable structure for transferring signals and picking up signals, regardless of whether the signals are electrical, magnetic, optical, pressure-based, physical, acoustic, etc., according to various embodiments. According to some embodiments, electrodes 110 of array 101 are configured to couple capacitively to a target location. In some embodiments, array 101 and physiological information generator 120 are disposed in a wearable device, such as a wearable data-capable band 170, which may include a housing that encapsulates, or substantially encapsulates, array 101 of electrodes 110. In operations, physiological information generator 120 can determine the bioelectric impedance (“bioimpedance”) of one or more types of tissues of a wearer to identify, measure, and monitor physiological characteristics. For example, a drive signal having a known amplitude and frequency can be applied to a user, from which a sink signal is received as bioimpedance signal. The bioimpedance signal is a measured signal that includes real and complex components. Examples of real components include extra-cellular and intra-cellular spaces of tissue, among other things, and examples of complex components include cellular membrane capacitance, among other things. Further, the measured bioimpedance signal can include real and/or complex components associated with arterial structures (e.g., arterial cells, etc.) and the presence (or absence) of blood pulsing through an arterial structure. In some examples, a heart rate signal, or other physiological signals, can be determined (i.e., recovered) from the measured bioimpedance signal by, for example, comparing the measured bioimpedance signal against the waveform of the drive signal to determine a phase delay (or shift) of the measured complex components.

Physiological information generator 120 is shown to include a sensor selector 122, a motion artifact reduction unit 124, and a physiological characteristic determinator 126. Sensor selector 122 is configured to select a subset of electrodes, and is further configured to use the selected subset of electrodes to acquire physiological characteristics, according to some embodiments. Examples of a subset of electrodes include subset 107, which is composed of electrodes 110d and 110e, and subset 105, which is composed of electrodes 110c, 110d and 110e. More or fewer electrodes can be used. Sensor selector 122 is configured to determine which one or more subsets of electrodes 110 (out of a number of subsets of electrodes 110) are adjacent to a target location. As used herein, the term “target location” can, for example, refer to a region in space from which a physiological characteristic can be determined. A target region can be adjacent to a source of the physiological characteristic, such as blood vessel 102, with which an impedance signal can be captured and analyzed to identify one or more physiological characteristics. The target region can reside in two-dimensional space, such as an area on the skin of a user adjacent to the source of the physiological characteristic, or in three-dimensional space, such as a volume that includes the source of the physiological characteristic. Sensor selector 122 operates to either drive a first signal via a selected subset to a target location, or receive a second signal from the target location, or both. The second signal includes data representing one or more physiological characteristics. For example, sensor selector 122 can configure electrode (“D”) 110b to operate as a drive electrode that drives a signal (e.g., an AC signal) into the target location, such as into the skin of a user, and can configure electrode (“S”) 110a to operate as a sink electrode (i.e., a receiver electrode) to receive a second signal from the target location, such as from the skin of the user. In this configuration, sensor selector 112 can drive a current signal via electrode (“D”) 110b into a target location to cause a current to pass through the target location to another electrode (“S”) 110a. In various examples, the target location can be adjacent to or can include blood vessel 102. Examples of blood vessel 102 include a radial artery, an ulnar artery, or any other blood vessel. Array 101 is not limited to being disposed adjacent blood vessel 102 in an arm, but can be disposed on any portion of a user's person (e.g., on an ankle, ear lobe, around a finger or on a fingertip, etc.). Note that each electrode 110 can be configured as either a driver or a sink electrode. Thus, electrode 110b is not limited to being a driver electrode and can be configured as a sink electrode in some implementations. As used herein, the term “sensor” can refer, for example, to a combination of one or more driver electrodes and one or more sink electrodes for determining one or more bioimpedance-related values and/or signals, according to some embodiments.

In some embodiments, sensor selector 122 can be configured to determine (periodically or aperiodically) whether the subset of electrodes 110a and 110b are optimal electrodes 110 for acquiring a sufficient representation of the one or more physiological characteristics from the second signal. To illustrate, consider that electrodes 110a and 110b may be displaced from the target location when, for instance, wearable device 170 is subject to a displacement in a plane substantially perpendicular to blood vessel 102. The displacement of electrodes 110a and 110b may increase the impedance (and/or reactance) of a current path between the electrodes 110a and 110b, or otherwise move those electrodes away from the target location far enough to degrade or attenuate the second signals retrieved therefrom. While electrodes 110a and 110b may be displaced from the target location, other electrodes are displaced to a position previously occupied by electrodes 110a and 110b (i.e., adjacent to the target location). For example, electrodes 110c and 110d may be displaced to a position adjacent to blood vessel 102. In this case, sensor selector 122 operates to determine an optimal subset of electrodes 110, such as electrodes 110c and 110d, to acquire the one or more physiological characteristics. Therefore, regardless of the displacement of wearable device 170 about blood vessel 102, sensor selector 122 can repeatedly determine an optimal subset of electrodes for extracting physiological characteristic information from adjacent a blood vessel. For example, sensor selector 122 can repeatedly test subsets in sequence (or in any other matter) to determine which one is disposed adjacent to a target location. For example, sensor selector 122 can select at least one of subset 109a, subset 109b, subset 109c, and other like subsets, as the subset from which to acquire physiological data.

According to some embodiments, array 101 of electrodes can be configured to acquire one or more physiological characteristics from multiple sources, such as multiple blood vessels. To illustrate, consider that, for example, blood vessel 102 is an ulnar artery adjacent electrodes 110a and 110b and a radial artery (not shown) is adjacent electrodes 110c and 110d. With multiple sources of physiological characteristic information being available, there are thus multiple target locations. Therefore, sensor selector 122 can select multiple subsets of electrodes 110, each of which is adjacent to one of a multiple number of target locations. Physiological information generator 120 then can use signal data from each of the multiple sources to confirm accuracy of data acquired, or to use one subset of electrodes (e.g., associated with a radial artery) when one or more other subsets of electrodes (e.g., associated with an ulnar artery) are unavailable.

Note that the second signal received into electrode 110a can be composed of a physiological-related signal component and a motion-related signal component, if array 101 is subject to motion. The motion-related component includes motion artifacts or noise induced into an electrode 110a. Motion artifact reduction unit 124 is configured to receive motion-related signals generated at one or more motion sensors 160, and is further configured to receive at least the motion-related signal component of the second signal. Motion artifact reduction unit 124 operates to eliminate the magnitude of the motion-related signal component, or to reduce the magnitude of the motion-related signal component relative to the magnitude of the physiological-related signal component, thereby yielding as an output the physiological-related signal component (or an approximation thereto). Thus, motion artifact reduction unit 124 can reduce the magnitude of the motion-related signal component (i.e., the motion artifact) by an amount associated with the motion-related signal generated by one or more accelerometers to yield the physiological-related signal component.

Physiological characteristic determinator 126 is configured to receive the physiological-related signal component of the second signal and is further configured to process (e.g., digitally) the signal data including one or more physiological characteristics to derive physiological signals, such as either a heart rate (“HR”) signal or a respiration signal, or both. For example, physiological characteristic determinator 126 is configured to amplify and/or filter the physiological-related component signals (e.g., at different frequency ranges) to extract certain physiological signals. According to various embodiments, a heart rate signal can include (or can be based on) a pulse wave. A pulse wave includes systolic components based on an initial pulse wave portion generated by a contracting heart, and diastolic components based on a reflected wave portion generated by the reflection of the initial pulse wave portion from other limbs. In some examples, an HR signal can include or otherwise relate to an electrocardiogram (“ECG”) signal. Physiological characteristic determinator 126 is further configured to calculate other physiological characteristics based on the acquired one or more physiological characteristics. Optionally, physiological characteristic determinator 126 can use other information to calculate or derive physiological characteristics. Examples of the other information include motion-related data, including the type of activity in which the user is engaged, such as running or sleep, location-related data, environmental-related data, such as temperature, atmospheric pressure, noise levels, etc., and any other type of sensor data, including stress-related levels and activity levels of the wearer.

In some cases, a motion sensor 160 can be disposed adjacent to the target location (not shown) to determine a physiological characteristic via motion data indicative of movement of blood vessel 102 through which blood pulses to identify a heart rate-related physiological characteristic. Motion data, therefore, can be used to supplement impedance determinations of to obtain the physiological characteristic.

Further, one or more motion sensors 160 can also be used to determine the orientation of wearable device 170, and relative movement of the same to determine or predict a target location. By predicting a target location, sensor selector 122 can use the predicted target location to begin the selection of optimal subsets of electrodes 110 in a manner that reduces the time to identify a target location.

In view of the foregoing, the functions and/or structures of array 101 of electrodes and physiological information generator 120, as well as their components, can facilitate the acquisition and derivation of physiological characteristics in situ—during which a user is engaged in physical activity that imparts motion on a wearable device, thereby exposing the array of electrodes to motion-related artifacts. Physiological information generator 120 is configured to dampen or otherwise negate the motion-related artifacts from the signals received from the target location, thereby facilitating the provision of heart-related activity and respiration activity to the wearer of wearable device 170 in real-time (or near real-time). As such, the wearer of wearable device 170 need not be stationary or otherwise interrupt an activity in which the wearer is engaged to acquire health-related information. Also, array 101 of electrodes 110 and physiological information generator 120 are configured to accommodate displacement or movement of wearable device 170 about, or relative to, one or more target locations. For example, if the wearer intentionally rotates wearable device 170 about, for example, the wrist of the user, then initial subsets of electrodes 110 adjacent to the target locations (i.e., before the rotation) are moved further away from the target location. As another example, the motion of the wearer (e.g., impact forces experienced during running) may cause wearable device 170 to travel about the wrist. As such, physiological information generator 120 is configured to determine repeatedly whether to select other subsets of electrodes 110 as optimal subsets of electrodes 110 for acquiring physiological characteristics. For example, physiological information generator 120 can be configured to cycle through multiple combinations of driver electrodes and sink electrodes (e.g., subsets 109a, 109b, 109c, etc.) to determine optimal subsets of electrodes. In some embodiments, electrodes 110 in array 101 facilitate physiological data capture irrespective of the gender of the wearer. For example, electrodes 110 can be disposed in array 101 to accommodate data collection of a male or female were irrespective of gender-specific physiological dimensions. In at least one embodiment, data representing the gender of the wearer can be accessible to assist physiological information generator 120 in selecting the optimal subsets of electrodes 110. While electrodes 110 are depicted as being equally-spaced, array 101 is not so limited. In some embodiments, electrodes 110 can be clustered more densely along portions of array 101 at which blood vessels 102 are more likely to be adjacent. For example, electrodes 110 may be clustered more densely at approximate portions 172 of wearable device 170, whereby approximate portions 172 are more likely to be adjacent a radial or ulnar artery than other portions. While wearable device 170 is shown to have an elliptical-like shape, it is not limited to such a shape and can have any shape.

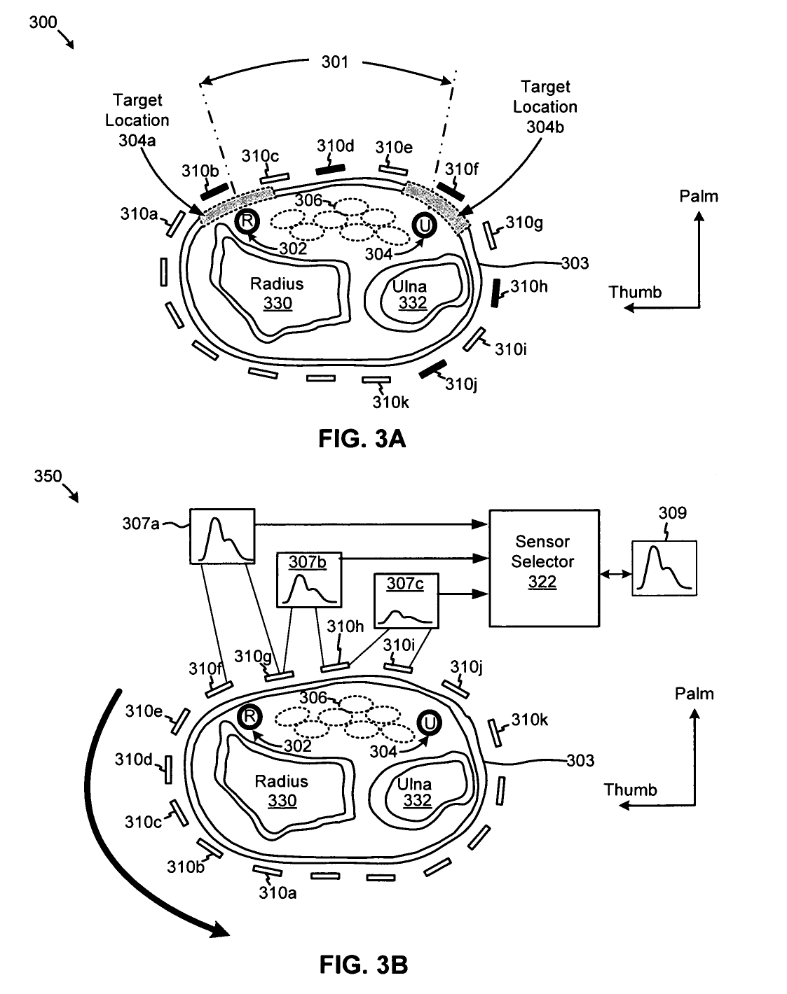

In some instances, a wearable device 170 can select multiple subsets of electrodes to enable data capture using a second subset adjacent to a second target location when a first subset adjacent a first target location is unavailable to capture data. For example, a portion of wearable device 170 including the first subset of electrodes 110 (initially adjacent to a first target location) may be displaced to a position farther away in a radial direction away from a blood vessel, such as depicted by a radial distance 392 of FIG. 3C from the skin of the wearer. That is, subset of electrodes 310a and 310b are displaced radially be distance 392. Further to FIG. 3C, the second subset of electrodes 310f and 310g adjacent to the second target location can be closer in a radial direction toward another blood vessel, and, thus, the second subset of electrodes can acquire physiological characteristics when the first subset of electrodes cannot. Referring back to FIG. 1A, array 101 of electrodes 110 facilitates a wearable device 170 that need not be affixed firmly to the wearer. That is, wearable device 170 can be attached to a portion of the wearer in a manner in which wearable device 170 can be displaced relative to a reference point affixed to the wearer and continue to acquire and generate information regarding physiological characteristics. In some examples, wearable device 170 can be described as being “loosely fitting” on or “floating” about a portion of the wearer, such as a wrist, whereby array 101 has sufficient sensors points from which to pick up physiological signals.

In addition, accelerometers 160 can be used to replace the implementation of subsets of electrodes to detect motion associated with pulsing blood flow, which, in turn, can be indicative of whether oxygen-rich blood is present or not present. Or, accelerometers 160 can be used to supplement the data generated by acquired one or more bioimpedance signals acquired by array 101. Accelerometers 160 can also be used to determine the orientation of wearable device 170 and relative movement of the same to determine or predict a target location. Sensor selector 122 can use the predicted target location to begin the selection of the optimal subsets of electrodes 110, which likely decreases the time to identify a target location. Electrodes 110 of array 101 can be disposed within a material constituting, for example, a housing, according to some embodiments. Therefore, electrodes 110 can be protected from the environment and, thus, need not be subject to corrosive elements. In some examples, one or more electrodes 110 can have at least a portion of a surface exposed. As electrodes 110 of array 101 are configured to couple capacitively to a target location, electrodes 110 thereby facilitate high impedance signal coupling so that the first and second signals can pass through fabric and hair. As such, electrodes 110 need not be limited to direct contact with the skin of a wearer. Further, array 101 of electrodes 110 need not circumscribe a limb or source of physiological characteristics. An array 101 can be linear in nature, or can configurable to include linear and curvilinear portions.

In some embodiments, wearable device 170 can be in communication (e.g., wired or wirelessly) with a mobile device 180, such as a mobile phone or computing device. In some cases, mobile device 180, or any networked computing device (not shown) in communication with wearable device 170 or mobile device 180, can provide at least some of the structures and/or functions of any of the features described herein. As depicted in FIG. 1A and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1A (or any subsequent figure) can represent one or more algorithms. Or, at least one of the elements can represent a portion of logic including a portion of hardware configured to provide constituent structures and/or functionalities.

For example, physiological information generator 120 and any of its one or more components, such as sensor selector 122, motion artifact reduction unit 124, and physiological characteristic determinator 126, can be implemented in one or more computing devices (i.e., any mobile computing device, such as a wearable device or mobile phone, whether worn or carried) that include one or more processors configured to execute one or more algorithms in memory. Thus, at least some of the elements in FIG. 1A (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, physiological information generator 120, including one or more components, such as sensor selector 122, motion artifact reduction unit 124, and physiological characteristic determinator 126, can be implemented in one or more computing devices that include one or more circuits. Thus, at least one of the elements in FIG. 1A (or any subsequent figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

FIGS. 1B to 1D illustrate examples of electrode arrays, according to some embodiments. Diagram 130 of FIG. 1B depicts an array 132 that includes sub-arrays 133a, 133b, and 133c of electrodes 110 that are configured to generate data that represent one or more characteristics associated with a user associated with array 132. In various embodiments, drive electrodes and sink electrodes can be disposed in the same sub-array or in different sub-arrays. Note that arrangements of sub-arrays 133a, 133b, and 133c can denote physical or spatial orientations and need not imply electrical, magnetic, or cooperative relationships among electrodes 110 within each sub-array. For example, drive electrode (“D”) 110f can be configured in sub-array 133a as a drive electrode to drive a signal to sink electrode (“S”) 110g in sub-array 133b. As another example, drive electrode (“D”) 110h can be configured in sub-array 133a to drive a signal to sink electrode (“S”) 110k in sub-array 133c. In some embodiments, distances between electrodes 110 in sub-arrays can vary at different regions, including a region in which the placement of electrode group 134 near blood vessel 102 is more probable relative to the placement of other electrodes near blood vessel 102. Electrode group 134 can include a higher density of electrodes 110 than other portions of array 132 as group 134 can be expected to be disposed adjacent blood vessel 102 more likely than other groups of electrodes 110. For example, an elliptical-shaped array (not shown) can be disposed in device 170 of FIG. 1A. Therefore, group 134 of electrodes is disposed at a region 172 of FIG. 1A, which is likely adjacent either a radial artery or an ulna artery. While three sub-arrays are shown, more or fewer are possible.

Referring to FIG. 1C, diagram 140 depicts an array 142 oriented at any angle (“θ”) 144 to an axial line coincident with or parallel to blood vessel 102. Therefore, an array 142 of electrodes need not be oriented orthogonally in each implementation; rather array 142 can be oriented at angles between 0 and 90 degrees, inclusive thereof. In a specific embodiment, an array 146 can be disposed parallel (or substantially parallel) to blood vessel 102a (or a portion thereof).

FIG. 1D is a diagram 150 depicting a wearable device 170a including a helically-shaped array 152 of electrodes disposed therein, whereby electrodes 110m and 110n can be configured as a pair of drive and sink electrodes. As shown, electrodes 110m and 110n substantially align in a direction parallel to an axis 151, which can represent a general direction of blood flow through a blood vessel.

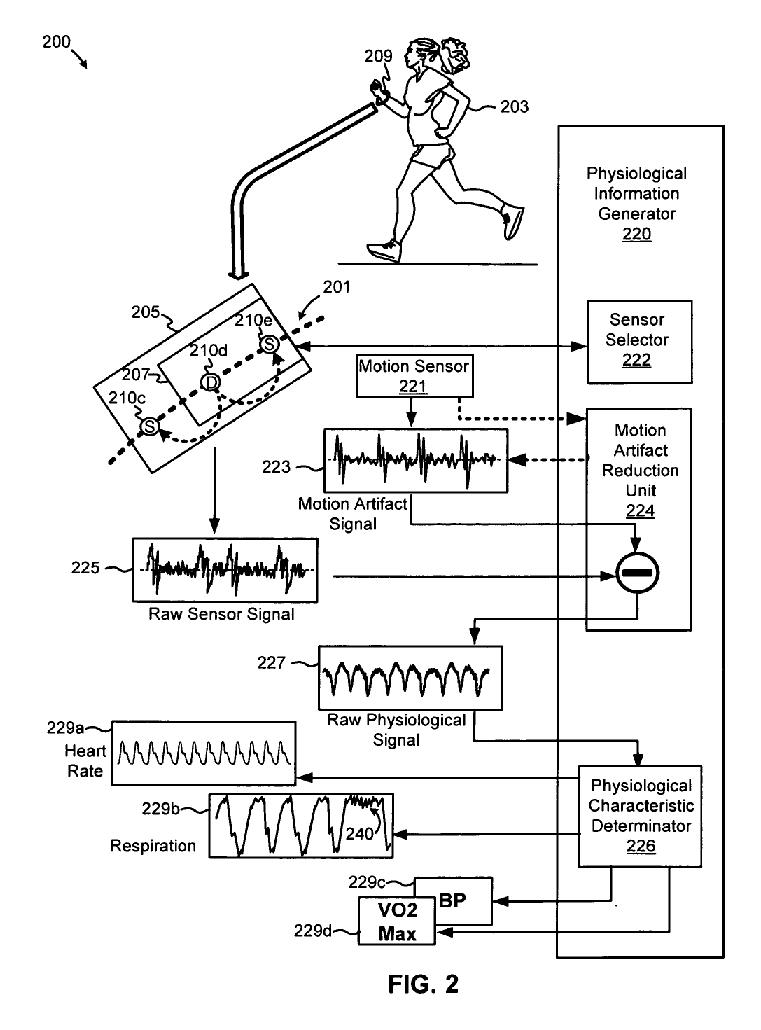

FIG. 2 is a functional diagram depicting a physiological information generator implemented in a wearable device, according to some embodiments. Functional diagram 200 depicts a user 203 wearing a wearable device 209, which includes a physiological information generator 220 configured to generate signals including data representing physiological characteristics. As shown, sensor selector 222 is configured to select a subset 205 of electrodes or a subset 207 of electrodes. Subset 205 of electrodes includes electrodes 210c, 210d, and 210e, and subset 207 of electrodes includes electrodes 210d and 210e. For purposes of illustration, consider that sensor selector 222 selects electrodes 210d and 210c as a subset of electrodes with which to capture physiological characteristics adjacent a target location. Sensor selector 222 applies an AC signal, as a first signal, into electrodes 210d to generate a sensor signal (“raw sensor signal”) 225, as a second signal, from electrode 210c. Sensor signal 222 includes a motion-related signal component and a physiological-related signal component. A motion sensor 221 is configured to capture generate a motion artifact signal 223 based on motion data representing motion experienced by wearable device 209 (or at least the electrodes). A motion artifact reduction unit 224 is configured to receive sensor signal 225 and motion artifact signal 223. Motion artifact reduction unit 224 operates to subtract motion artifact signal 223 from sensor signal 225 to yield the physiological-related signal component (or an approximation thereof) as a raw physiological signal 227. In some examples, raw physiological signal 227 represents an unamplified, unfiltered signal including data representative of one or more physiological characteristics. In some embodiments, motion sensor 221 generates motion signals, such as accelerometer signals. These signals are provided to motion artifact reduction unit 224 (e.g., via dashed lines as shown), which, in turn, is configured to determine motion artifact signal 223. In some embodiments, motion artifact signal 223 represents motion included or embodied within raw sensor signal 225 (e.g., with physiological signal(s)). Thus, a motion artifact signal can describe a motion signal, whether sensed by a motion sensor or integrated with one or more physiological signals. A physiological characteristic determinator 226 is configured to receive raw physiological signal 227 to amplify and/or filter different physiological signal components from raw physiological signal 227. For example, raw physiological signal 227 may include a respiration signal modulated on (or in association with) a heart rate (“HR”) signal. Regardless, physiological characteristic determinator 226 is configured to perform digital signal processing to generate a heart rate (“HR”) signal 229a and/or a respiration signal 229b. Portion 240 of respiration signal 229b represents an impedance signal due to cardiac activity, at least in some instances. Further, physiological characteristic determinator 226 is configured to use either HR signal 229a or a respiration signal 229b, or both, to derive other physiological characteristics, such as blood pressure data (“BP”) 229c, a maximal oxygen consumption (“VO2 max”) 229d, or any other physiological characteristic.

Physiological characteristic determinator 226 can derive other physiological characteristics using other data generated or accessible by wearable device 209, such as the type of activity the wear is engaged, environmental factors, such as temperature, location, etc., whether the wearer is subject to any chronic illnesses or conditions, and any other health or wellness-related information. For example, if the wearer is diabetic or has Parkinson's disease, motion sensor 221 can be used to detect tremors related to the wearer's ailment. With the detection of small, but rapid movements of a wearable device that coincide with a change in heart rate (e.g., a change in an HR signal) and/or breathing, physiological information generator 220 may generate data (e.g., an alarm) indicating that the wearer is experiencing tremors. For a diabetic, the wearer may experience shakiness because the blood-sugar level is extremely low (e.g., it drops below a range of 38 to 42 mg/dl). Below these levels, the brain may become unable to control the body. Moreover, if the arms of a wearer shakes with sufficient motion to displace a subset of electrodes from being adjacent a target location, the array of electrodes, as described herein, facilitates continued monitoring of a heart rate by repeatedly selecting subsets of electrodes that are positioned optimally (e.g., adjacent a target location) for receiving robust and accurate physiological-related signals.

FIGS. 3A to 3C are cross-sectional views depicting arrays of electrodes including subsets of electrodes adjacent an arm portion of a wearer, according to some embodiments. Diagram 300 of FIG. 3A depicts an array of electrodes arranged about, for example, a wrist of a wearer. In this cross-sectional view, an array of electrodes includes electrodes 310a, 310b, 310c, 310d, 310e, 310f, 310g, 310h, 310i, 310j, and 310k, among others, arranged about wrist 303 (or the forearm). The cross-sectional view of wrist 303 also depicts a radius bone 330, an ulna bone 332, flexor muscles/ligaments 306, a radial artery (“R”) 302, and an ulna artery (“U”) 304. Radial artery 302 is at a distance 301 (regardless of whether linear or angular) from ulna artery 304. Distance 301 may be different, on average, for different genders, based on male and female anatomical structures. Notably, the array of electrodes can obviate specific placement of electrodes due to different anatomical structures based on gender, preference of the wearer, issues associated with contact (e.g., contact alignment), or any other issue that affects placement of electrode that otherwise may not be optimal. To effect appropriate electrode selection, a sensor selector, as described herein, can use gender-related information (e.g., whether the wearer is male or female) to predict positions of subsets of electrodes such that they are adjacent (or substantially adjacent) to one or more target locations 304a and 304b. Target locations 304a and 304b represent optimal areas (or volumes) at which to measure, monitor and capture data related to bioimpedances. In particular, target location 304a represents an optimal area adjacent radial artery 302 to pick up bioimpedance signals, whereas target location 304b represents another optimal area adjacent ulna artery 304 to pick up other bioimpedance signals.

To illustrate the resiliency of a wearable device to maintain an ability to monitor physiological characteristics over one or more displacements of the wearable device (e.g., around or along wrist 303), consider that a sensor selector configures initially electrodes 310b, 310d, 310f, 310h, and 310j as driver electrodes and electrodes 310a, 310c, 310e 310g, 310i, and 310k as sink electrodes. Further consider that the sensor selector identifies a first subset of electrodes that includes electrodes 310b and 310c as a first optimal subset, and also identifies a second subset of electrodes that include electrodes 310f and 310g as a second optimal subset. Note that electrodes 310b and 310c are adjacent target location 304a and electrodes 310f and 310g are adjacent to target location 304b. These subsets are used to periodically (or aperiodically) monitor the signals from electrodes 310c and 310g, until the first and second subsets are no longer optimal (e.g., when movement of the wearable device displaces the subsets relative to the target locations). Note that the functionality of driver and sink electrodes for electrodes 310b, 310c, 310f, and 310g can be reversed (e.g., electrodes 310a and 310g can be configured as drive electrodes).

FIG. 3B depicts an array of FIG. 3A being displaced from an initial position, according to some examples. In particular, diagram 350 depicts that electrodes 310f and 310g are displaced to a location adjacent radial artery 302 and electrodes 310j and 310k are displaced to a location adjacent ulna artery 304. According to some embodiments, a sensor selector 322 is configured to test subsets of electrodes to determine at least one subset, such as electrodes 310f and 310, being located adjacent to a target location (next to radial artery 302). To identify electrodes 310f and 310g as an optimal subset, sensor selector 322 is configured to apply drive signals to the drive electrodes to generate a number of data samples, such as data samples 307a, 307b, and 307c. In this example, each data sample represents a portion of a physiological characteristic, such as a portion of an HR signal. Sensor selector 322 operates to compare the data samples against a profile 309 to determine which of data samples 307a, 307b, and 307c best fits or is comparable to a predefined set of data represented by profile data 309. Profile data 309, in this example, represents an expected HR portion or thresholds indicating a best match. Also, profile data 309 can represent the most robust and accurate HR portion measured during the sensor selection mode relative to all other data samples (e.g., data sample 307a is stored as profile data 309 until, and if, another data sample provides a more robust and/or accurate data sample). As shown, data sample 307a substantially matches profile data 309, whereas data samples 307b and 307c are increasingly attenuated as distances increase away from radial artery 302. Therefore, sensor selector 322 identifies electrodes 310f and 310g as an optimal subset and can use this subset in data capture mode to monitor (e.g., continuously) the physiological characteristics of the wearer. Note that the nature of data samples 307a, 307b, and 307c as portions of an HR signal is for purposes of explanation and is not intended to be limiting. Data samples 307a, 307b, and 307c need not be portions of a waveform or signal, and need not be limited to an HR signal. Rather, data samples 307a, 307b, and 307c can relate to a respiration signal, a raw sensor signal, a raw physiological signal, or any other signal. Data samples 307a, 307b, and 307c can represent a measured signal attribute, such as magnitude or amplitude, against which profile data 309 is matched. In some cases, an optimal subset of electrodes can be associated with a least amount of impedance and/or reactance (e.g., over a period of time) when applying a first signal (e.g., a drive signal) to a target location.

FIG. 3C depicts an array of electrodes of FIG. 3A oriented differently due to a change in orientation of a wrist of a wearer, according to some examples. In this example, the array of electrodes is shown to be disposed in a wearable device 371, which has an outer surface 374 and an inner surface 372. In some embodiments, wearable device 371 can be configured to “loosely fit” around the wrist, thereby enabling rotation about the wrist. In some cases, a portion of wearable devices 371 (and corresponding electrodes 310a and 310b) are subject to gravity (“G”) 390, which pulls the portion away from wrist 303, thereby forming a gap 376. Gap 376, in turn, causes inner surface 372 and electrodes 310a and 310b to be displaced radially by a radial distance 392 (i.e., in a radial direction away from wrist 303). Gap 376, in some cases, can be an air gap. Radial distance 392, at least in some cases, may impact electrodes 310a and 310b and the ability to receive signals adjacent to radial artery 302. Regardless, electrodes 310f and 310g are positioned in another portion of wearable device 371 and can be used to receive signals adjacent to ulna artery 304 in cooperation with, or instead of, electrodes 310a and 310b. Therefore, electrodes 310f and 310g (or any other subset of electrodes) can provide redundant data capturing capabilities should other subsets be unavailable.

Next, consider that sensor selector 322 of FIG. 3B is configured to determine a position of electrodes 310f and 310g (e.g., on the wearable device 371) relative to a direction of gravity 390. A motion sensor (not shown) can determine relative movements of the position of electrodes 310f and 310g over any number of movements in either a clockwise direction (“dCW”) or a counterclockwise direction (“dCCW”). As wearable device 371 need not be affixed firmly to wrist 303, at least in some examples, the position of electrodes 310f and 310g may “slip” relative to the position of ulna artery 304. In one embodiment, sensor selector 322 can be configured to determine whether another subset of electrodes are optimal, if electrodes 310f and 310g are displaced farther away than a more suitable subset. In sensor selecting mode, sensor selector 322 is configured to select another subset, if necessary, by beginning the capture of data samples at electrodes 310f and 310g and progressing to other nearby subsets to either confirm the initial selection of electrodes 310f and 310g or to select another subset. In this manner, the identification of the optimal subset may be determined in less time than if the selection process is performed otherwise (e.g., beginning at a specific subset regardless of the position of the last known target location).

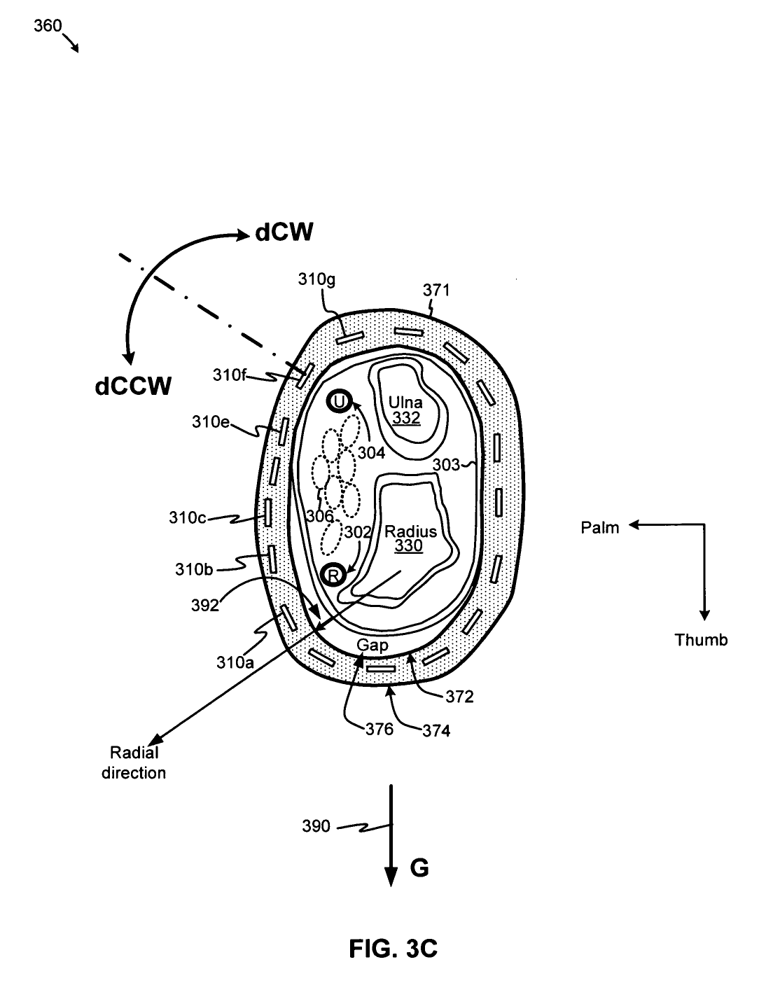

FIG. 4 depicts a portion of an array of electrodes disposed within a housing material of a wearable device, according to some embodiments. Diagram 400 depicts electrodes 410a and 410b disposed in a wearable device 401, which has an outer surface 402 and an inner surface 404. In some embodiments, wearable device 401 includes a material in which electrodes 410a and 410b can be encapsulated in a material to reduce or eliminate exposure to corrosive elements in the environment external to wearable device 401. Therefore, material 420 is disposed between the surfaces of electrodes 410a and 410b and inner surface 404. Driver electrodes are capacitively coupled to skin 405 to transmit high impedance signals, such as a current signal, over distance (“d”) 422 through the material, and, optionally, through fabric 406 or hair into skin 405 of the wearer. Also, the current signal can be driven through an air gap (“AG”) 424 between inner surface 404 and skin 405. Note that in some implementations, electrodes 410a and 410b can be exposed (or partially exposed) out through inner surface 404. In some embodiments, electrodes 410a and 410b can be coupled via conductive materials, such as conductive polymers or the like, to the external environment of wearable device 401.

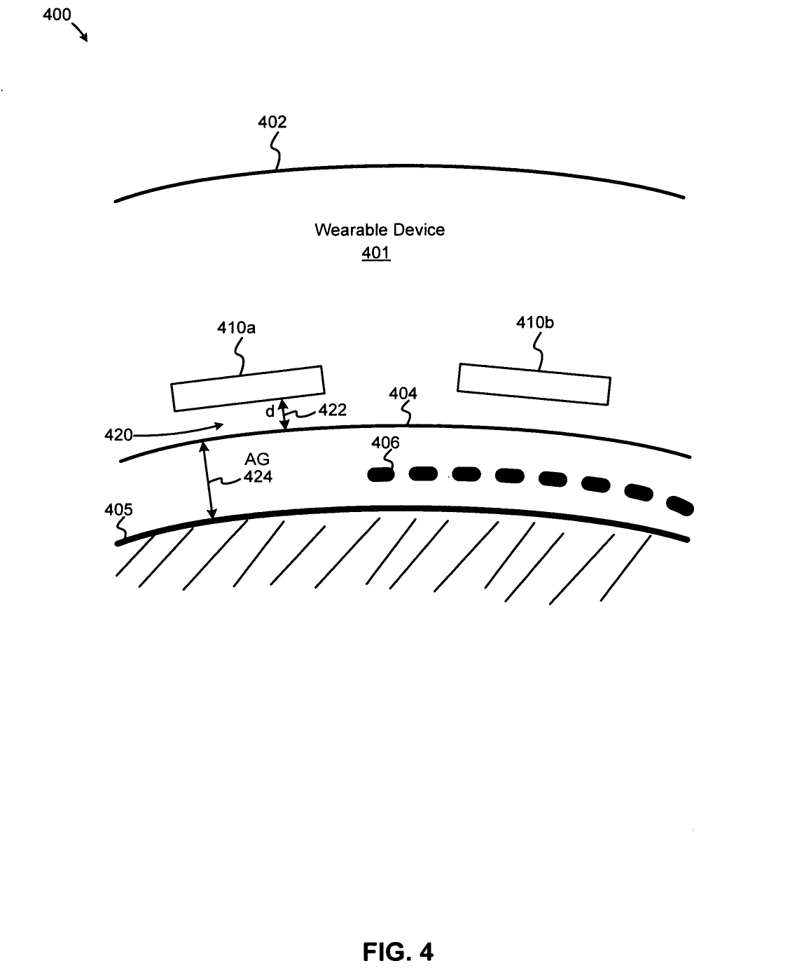

FIG. 5 depicts an example of a physiological information generator, according to some embodiments. Diagram 500 depicts an array 501 of electrodes 510 that can be disposed in a wearable device. A physiological information generator can include one or more of a sensor selector 522, an accelerometer 540 for generating motion data, a motion artifact reduction unit 524, and a physiological characteristic determinator 526. Sensor selector 522 includes a signal controller 530, a multiplexer 501 (or equivalent switching mechanism), a signal driver 532, a signal receiver 534, a motion determinator 536, and a target location determinator 538. Sensor selector 522 is configured to operate in at least two modes. First, sensor selector 522 can select a subset of electrodes in a sensor select mode of operation. Second, sensor selector 522 can use a selected subset of electrodes to acquire physiological characteristics, such as in a data capture mode of operation, according to some embodiments. In sensor select mode, signal controller 530 is configured to serially (or in parallel) configure subsets of electrodes as driver electrodes and sink electrodes, and to cause multiplexer 501 to select subsets of electrodes 510. In this mode, signal driver 532 applies a drive signal via multiplexer 501 to a selected subset of electrodes, from which signal receiver 534 receives via multiplexer 501 a sensor signal. Signal controller 530 acquires a data sample for the subset under selection, and then selects another subset of electrodes 510. Signal controller 530 repeats the capture of data samples, and is configured to determine an optimal subset of electrodes for monitoring purposes. Then, sensor selector 522 can operate in the data capture mode of operation in which sensor selector 522 continuously (or substantially continuously) captures sensor signal data from at least one selected subset of electrodes 501 to identify physiological characteristics in real time (or in near real-time).

In some embodiments, a target location determinator 538 is configured to initiate the above-described sensor selection mode to determine a subset of electrodes 510 adjacent a target location. Further, target location determinator 538 can also track displacements of a wearable device in which array 501 resides based on motion data from accelerometer 540. For example, target location determinator 538 can be configured to determine an optimal subset if the initially-selected electrodes are displaced farther away from the target location. In sensor selecting mode, target location determinator 538 can be configured to select another subset, if necessary, by beginning the capture of data samples at electrodes for the last known subset adjacent to the target location, and progressing to other nearby subsets to either confirm the initial selection of electrodes or to select another subset. In some examples, orientation of the wearable device, based on accelerometer data (e.g., a direction of gravity), also can be used to select a subset of electrodes 501 for evaluation as an optimal subset. Motion determinator 536 is configured to detect whether there is an amount of motion associated with a displacement of the wearable device. As such, motion determinator 536 can detect motion and generate a signal to indicate that the wearable device has been displaced, after which signal controller 530 can determine the selection of a new subset that is more closely situated near a blood vessel than other subsets, for example. Also, motion determinator 536 can cause signal controller 530 to disable data capturing during periods of extreme motion (e.g., during which relatively large amounts of motion artifacts may be present) and to enable data capturing during moments when there is less than an extreme amount of motion (e.g., when a tennis player pauses before serving). Data repository 542 can include data representing the gender of the wearer, which is accessible by signal controller 530 in determining the electrodes in a subset.

In some embodiments, signal driver 532 may be a constant current source including an operational amplifier configured as an amplifier to generate, for example, 100 μA of alternating current (“AC”) at various frequencies, such as 50 kHz. Note that signal driver 532 can deliver any magnitude of AC at any frequency or combinations of frequencies (e.g., a signal composed of multiple frequencies). For example, signal driver 532 can generate magnitudes (or amplitudes), such as between 50 μA and 200 μA, as an example. Also, signal driver 532 can generate AC signals at frequencies from below 10 kHz to 550 kHz, or greater. According to some embodiments, multiple frequencies may be used as drive signals either individually or combined into a signal composed of the multiple frequencies. In some embodiments, signal receiver 534 may include a differential amplifier and a gain amplifier, both of which can include operational amplifiers.

Motion artifact reduction unit 524 is configured to subtract motion artifacts from a raw sensor signal received into signal receiver 534 to yield the physiological-related signal components for input into physiological characteristic determinator 526. Physiological characteristic determinator 526 can include one or more filters to extract one or more physiological signals from the raw physiological signal that is output from motion artifact reduction unit 524. A first filter can be configured for filtering frequencies for example, between 0.8 Hz and 3 Hz to extract an HR signal, and a second filter can be configured for filtering frequencies between 0 Hz and 0.5 Hz to extract a respiration signal from the physiological-related signal component. Physiological characteristic determinator 526 includes a biocharacteristic calculator that is configured to calculate physiological characteristics 550, such as VO2 max, based on extracted signals from array 501.

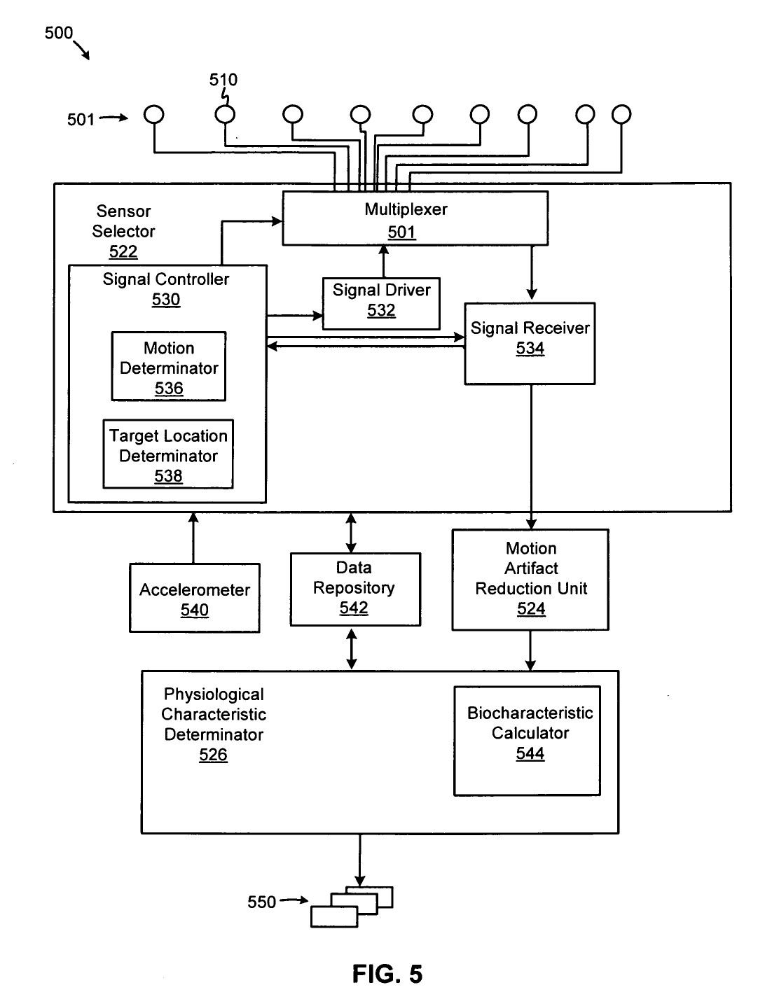

FIG. 6 is an example flow diagram for selecting a sensor, according to some embodiments. At 602, flow 600 provides for the selection of a first subset of electrodes and the selection of a second subset of electrodes in a select sensor mode. At 604, one of the first and second subset of electrodes is selected as a drive electrode and the other of the first and second subset of electrodes is selected as a sink electrode. In particular, the first subset of electrodes can, for example, include one or more drive electrodes, and the second subset of electrodes can include one or more sink electrodes. At 606, one or more data samples are captured, the data samples representing portions of a measured signal (or values thereof). Based on a determination that one of the data samples is indicative of a subset of electrodes adjacent a target location, the electrodes of the optimal subset are identified at 608. At 610, the identified electrodes are selected to capture signals including physiological-relate components. While there is no detected motion at 612, flow 600 moves to 616 to capture, for example, heart and respiration data continuously. When motion is detected at 612, data capture may continue. But flow 600 moves to 614 to determine whether to apply a predicted target location. In some cases, a predicted target location is based on the initial target location (e.g., relative to the initially-determined subset of electrodes), with subsequent calculations based on amounts and directions of displacement, based on accelerometer data, to predict a new target location. One or more motion sensors can be used to determine the orientation of a wearable device, and relative movement of the same (e.g., over a period of time or between events), to determine or predict a target location. Or, the predicted target location can refer to the last known target location and/or subset of electrodes. At 618, electrodes are selected based on the predicted target location for confirming whether the previously-selected subset of electrodes are optimal, or whether a new, optimal subset is to be determined as flow 600 moves back to 602.

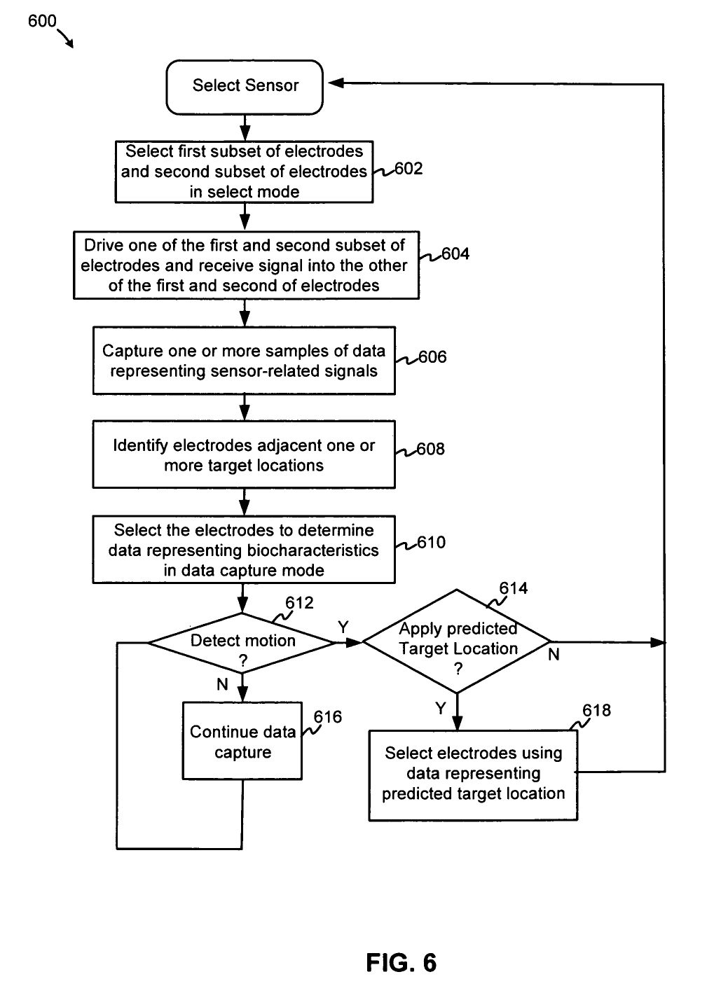

FIG. 7 is an example flow diagram for determining physiological characteristics using a wearable device with arrayed electrodes, according to some embodiments. At 702, flow 700 provides for the selection of a sensor in sensor select mode, the sensor including, for example, two or more electrodes. At 704, sensor signal data is captured in data capture mode. At 706, motion-related artifacts can be reduced or eliminated from the sensor signal to yield a physiological-related signal component. One or more physiological characteristics can be identified at 708, for example, after digitally processing the physiological-related signal component. At 710, one or more physiological characteristics can be calculated based on the data signals extracted at 708. Examples of calculated physiological characteristics include maximal oxygen consumption (“VO2 max”).

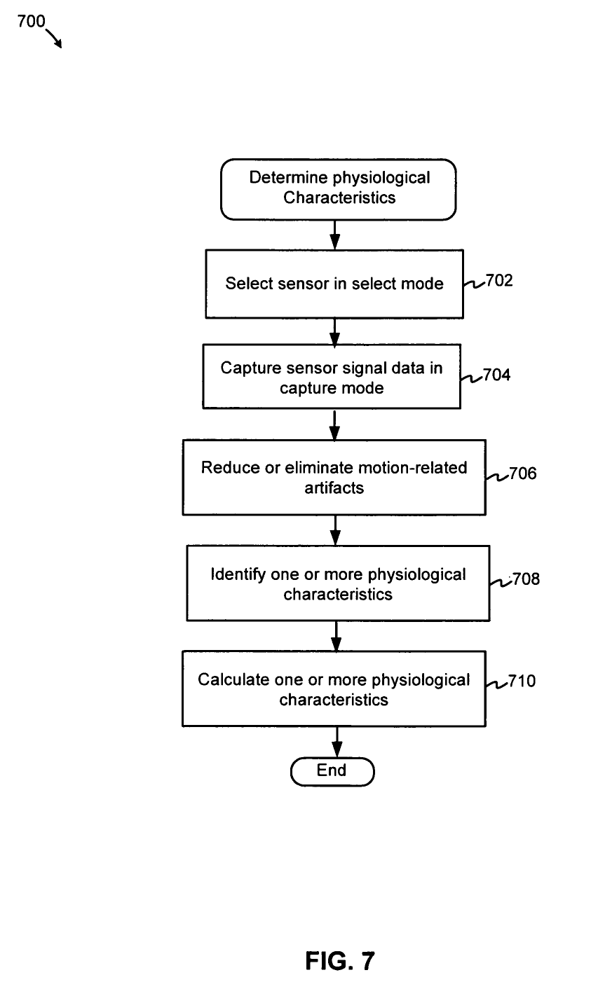

FIG. 8 illustrates an exemplary computing platform disposed in a wearable device in accordance with various embodiments. In some examples, computing platform 800 may be used to implement computer programs, applications, methods, processes, algorithms, or other software to perform the above-described techniques. Computing platform 800 includes a bus 802 or other communication mechanism for communicating information, which interconnects subsystems and devices, such as processor 804, system memory 806 (e.g., RAM, etc.), storage device 808 (e.g., ROM, etc.), a communication interface 813 (e.g., an Ethernet or wireless controller, a Bluetooth controller, etc.) to facilitate communications via a port on communication link 821 to communicate, for example, with a computing device, including mobile computing and/or communication devices with processors. Processor 804 can be implemented with one or more central processing units (“CPUs”), such as those manufactured by Intel® Corporation, or one or more virtual processors, as well as any combination of CPUs and virtual processors. Computing platform 800 exchanges data representing inputs and outputs via input-and-output devices 801, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 800 performs specific operations by processor 804 executing one or more sequences of one or more instructions stored in system memory 806, and computing platform 800 can be implemented in a client-server arrangement, peer-to-peer arrangement, or as any mobile computing device, including smart phones and the like. Such instructions or data may be read into system memory 806 from another computer readable medium, such as storage device 808. In some examples, hard-wired circuitry may be used in place of or in combination with software instructions for implementation. Instructions may be embedded in software or firmware. The term “computer readable medium” refers to any tangible medium that participates in providing instructions to processor 804 for execution. Such a medium may take many forms, including but not limited to, non-volatile media and volatile media. Non-volatile media includes, for example, optical or magnetic disks and the like. Volatile media includes dynamic memory, such as system memory 806.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 802 for transmitting a computer data signal.

In some examples, execution of the sequences of instructions may be performed by computing platform 800. According to some examples, computing platform 800 can be coupled by communication link 821 (e.g., a wired network, such as LAN, PSTN, or any wireless network) to any other processor to perform the sequence of instructions in coordination with (or asynchronous to) one another. Computing platform 800 may transmit and receive messages, data, and instructions, including program code (e.g., application code) through communication link 821 and communication interface 813. Received program code may be executed by processor 804 as it is received, and/or stored in memory 806 or other non-volatile storage for later execution.

In the example shown, system memory 806 can include various modules that include executable instructions to implement functionalities described herein. In the example shown, system memory 806 includes a physiological information generator module 854 configured to implement determine physiological information relating to a user that is wearing a wearable device. Physiological information generator module 854 can include a sensor selector module 856, a motion artifact reduction unit module 858, and a physiological characteristic determinator 859, any of which can be configured to provide one or more functions described herein.

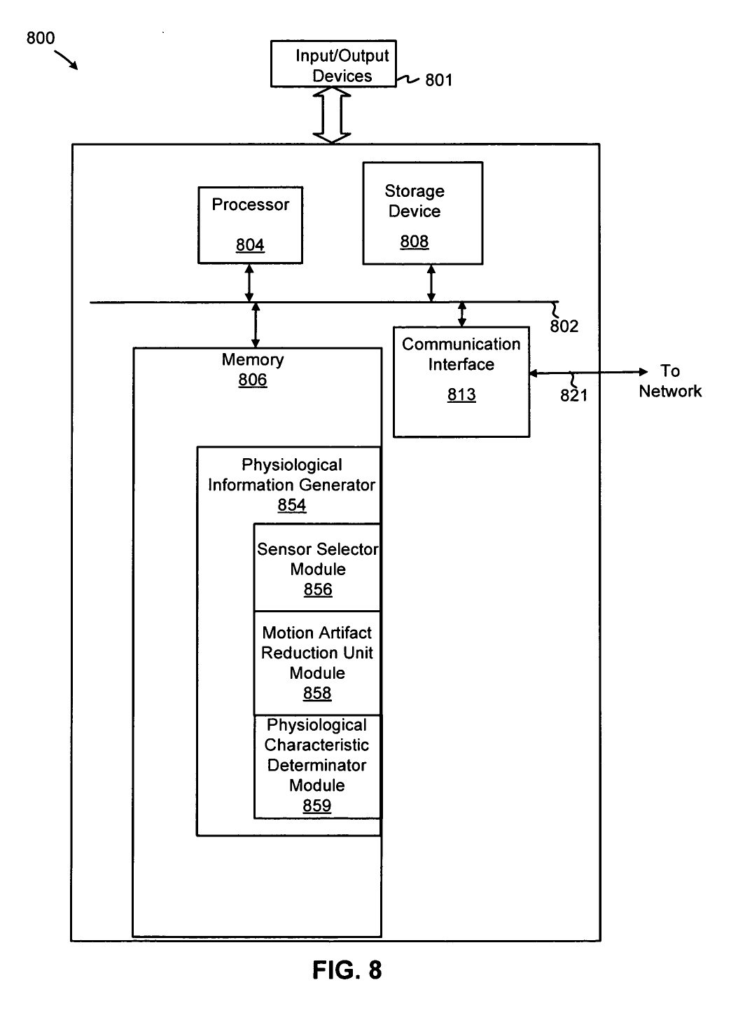

FIG. 9 depicts the physiological signal extractor, according to some embodiments. Diagram 900 depicts a motion artifact reduction unit 924 including a physiological signal extractor 936. In some embodiments, motion artifact reduction unit 924 can be disposed in or attached to a wearable device 909, which can be configured to attached to or otherwise be worn by user 903. As shown, user 903 is running or jogging, whereby movement of the limbs of user 903 imparts forces that cause wearable device 909 to experience motion. Motion artifact reduction unit 924 is configured to receive a sensor signal (“Raw Sensor Signal”) 925, and is further configured to reduce or negate motion artifacts accompanying, or mixed with, physiological signals due to motion-related noise that otherwise affects sensor signal 925. Further to diagram 900, a signal receiver 934 is coupled to a sensor including, for example, one or more electrodes. Examples of such electrodes include electrode 910a and electrode 910b. In some embodiments, signal receiver 934 includes similar structure and/or functionality as signal receiver 534 of FIG. 5. In operation, signal receiver 934 is configured to receive one or more AC current signals, such as high impedance signals, as bioimpedance-related signals. Signal receiver 934 can include differential amplifiers, gain amplifiers, or any other operational amplifier configured to receive, adapt (e.g., amplify), and transmit sensor signal 925 to motion artifact reduction unit 924.

In some embodiments, signal receiver 934 is configured to receive electrical signals representing acoustic-related information from a microphone 911. An example of the acoustic-related information includes data representing a heartbeat or a heart rate as sensed by microphone 911, such that sensor signal 925 can be an electrical signal derived from acoustic energy associated with a sensed physiological signal, such as a pulse wave or heartbeat. Wearable device 909 can include microphone 911 configured to contact (or to be positioned adjacent to) the skin of the wearer, whereby microphone 911 is adapted to receive sound and acoustic energy generated by the wearer (e.g., the source of sounds associated with physiological information). Microphone 911 can also be disposed in wearable device 909. According to some embodiments, microphone 911 can be implemented as a skin surface microphone (“SSM”), or a portion thereof, according to some embodiments. An SSM can be an acoustic microphone configured to enable it to respond to acoustic energy originating from human tissue rather than airborne acoustic sources. As such, an SSM facilitates relatively accurate detection of physiological signals through a medium for which the SSM can be adapted (e.g., relative to the acoustic impedance of human tissue). Examples of SSM structures in which piezoelectric sensors can be implemented (e.g., rather than a diaphragm) are described in U.S. patent application Ser. No. 11/199,856, filed on Aug. 8, 2005, and U.S. patent application Ser. No. 13/672,398, filed on Nov. 8, 2012, both of which are incorporated by reference. As used herein, the term human tissue can refer to, at least in some examples, as skin, muscle, blood, or other tissue. In some embodiments, a piezoelectric sensor can constitute an SSM. Data representing sensor signal 925 can include acoustic signal information received from an SSM or other microphone, according to some examples.

According to some embodiments, physiological signal extractor 936 is configured to receive sensor signal 925 and data representing sensing information 915 from another, secondary sensor 913. In some examples, sensor 913 is a motion sensor (e.g., an accelerometer) configured to sense accelerations in one or more axes and generates motion signals indicating an amount of motion and/or acceleration. Note, however, that sensor 913 need not be so limited and can be any other sensor. Examples of suitable sensors are disclosed in U.S. Non-Provisional patent application Ser. No. 13/492,857, filed on Jun. 9, 2012, which is incorporated by reference. Further, physiological signal extractor 936 is configured to operate to identify a pattern (e.g., a motion “signature”), based on motion signal data generated by sensor 913, that can used to decompose sensor signal 925 into motion signal components 937a and physiological signal components 937b. As shown, motion signal components 937a and physiological signal components 937b can correspondingly be used by motion artifact reduction unit 924, or any other structure and/or function described herein, to form motion data 930 and one or more physiological data signals, such as physiological characteristic signals 940, 942, and 944. Physiological characteristic determinator 926 is configured to receive physiological signal components 937b of a raw physiological signal, and to filter different physiological signal components to form physiological characteristic signal(s). For example, physiological characteristic determinator 926 can be configured to analyze the physiological signal components to determine a physiological characteristic, such as a heartbeat, heart rate, pulse wave, respiration rate, a Mayer wave, and other like physiological characteristic. Physiological characteristic determinator 926 is also configured to generate a physiological characteristic signal that includes data representing the physiological characteristic during one or more portions of a time interval during which motion is present. Examples of physiological characteristic signals include data representing one or more of a heart rate 940, a respiration rate 942, Mayer wave frequencies 944, and any other sensed characteristic, such as a galvanic skin response (“GSR”) or skin conductance. Note that the term “heart rate” can refer, at least in some embodiments, to any heart-related physiological signal, including, but not limited to, heart beats, heart beats per minute (“bpm”), pulse, and the like. In some examples, the term “heart rate” can refer also to heart rate variability (“HRV”), which describes the variation of a time interval between heartbeats. HRV describes a variation in the beat to beat interval and can be described in terms of frequency components (e.g., low frequency and high frequency components), at least in some cases.

In view of the foregoing, the functions and/or structures of motion artifact reduction unit 924, as well as its components and/or neighboring components, can facilitate the extraction and derivation of physiological characteristics in situ—during which a user is engaged in physical activity that imparts motion on a wearable device, whereby biometric sensors, such as electrodes, may receive bioimpedance sensor signals that are exposed to, or include, motion-related artifacts. For example, physiological signal extractor 936 can be configured to receive the sensor signal that includes data representing physical physiological characteristics during one or more portions of the time interval in which the wearable devices is in motion. A user 903 need not be required to remain immobile to determine physiological signal characteristic signals. Therefore, user 903 can receive heart rate information, respiration information, and other physiological information during physical activity or during periods of time in which user 903 is substantially or relatively active. Further, according to various embodiments, physiological signal extractor 936 facilitates the sensing of physiological characteristic signals at a distal end of a limb or appendage, such as at a wrist, of user 903. Therefore, various implementations of motion artifact reduction unit 924 can enable the detection of physiological signal at the extremities of user 903, with minimal or reduced effects of motion-related artifacts and their influence on the desired measured physiological signal. By facilitating the detection of physiological signals at the extremities, wearable device 909 can assist user 903 to detect oncoming ailments or conditions of the person's body (e.g., oncoming tremors, states of sleep, etc.) relative to other portions of the person's body, such as proximal portions of a limb or appendage.

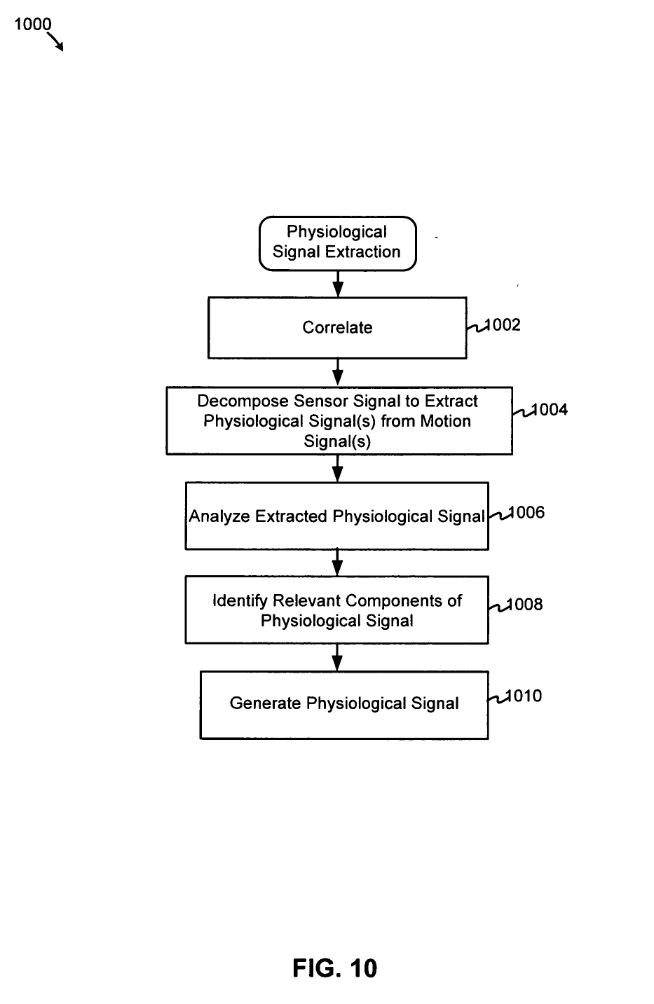

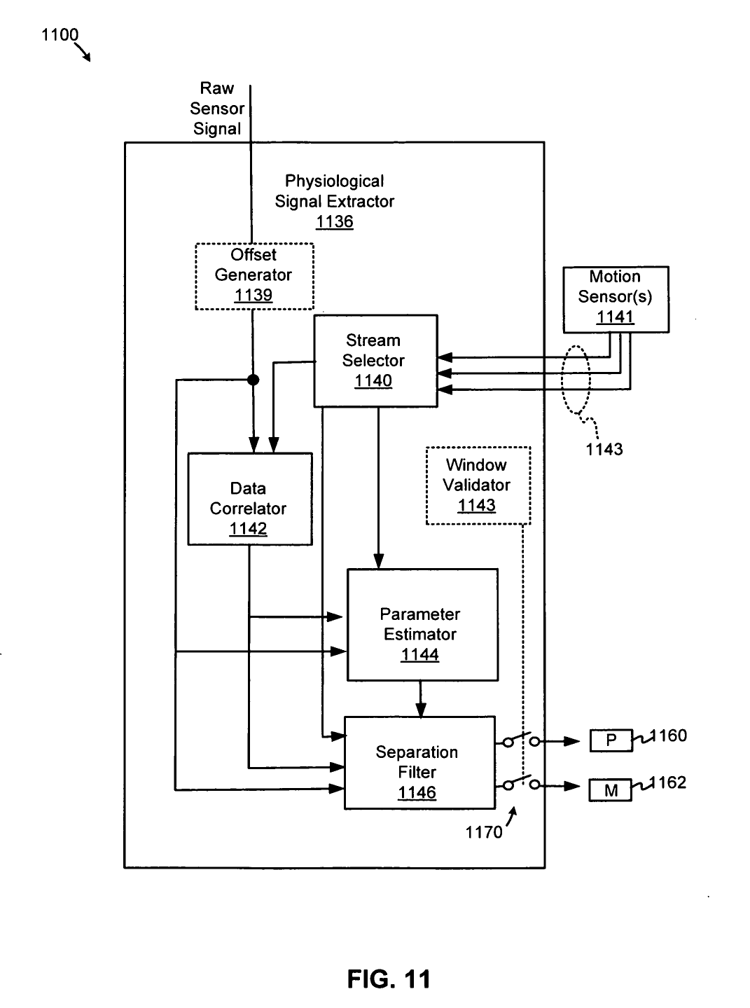

In accordance with some embodiments, physiological signal extractor 936 can include an offset generator, which is not shown. An offset generator can be configured to determine an amount of motion that is associated with the motion sensor signal, such as an accelerometer signal, and to adjust the dynamic range of operation of an amplifier, where the amplifier is configured to receive a sensor signal responsive to the amount of motion. An example of such an amplifier is an operational amplifier configured as a front-end amplifier to enhance, for example, the signal-to-noise ratio. In situations in which the motion related artifacts induce a rapidly-increasing amplitude onto the sensor signal, the amplifier may drive into saturation, which, in turn, causes clipping of the output of the amplifier. The offset generator also is configured to apply in offset value to an amplifier to modify the dynamic range of the amplifier so as to reduce or negate large magnitudes of motion artifacts that may otherwise influence the amplitude of the sensor signal. Examples of an offset generator are described in relation to FIG. 12. In some embodiments, physiological signal extractor 936 can include a window validator configured to determine durations (i.e., a valid window of time) in which sensor signal data can be predicted to be valid (i.e., durations in which the magnitude of motion-related artifacts signals likely do not influence the physiological signals). An example of a window validator is described in FIG. 11.

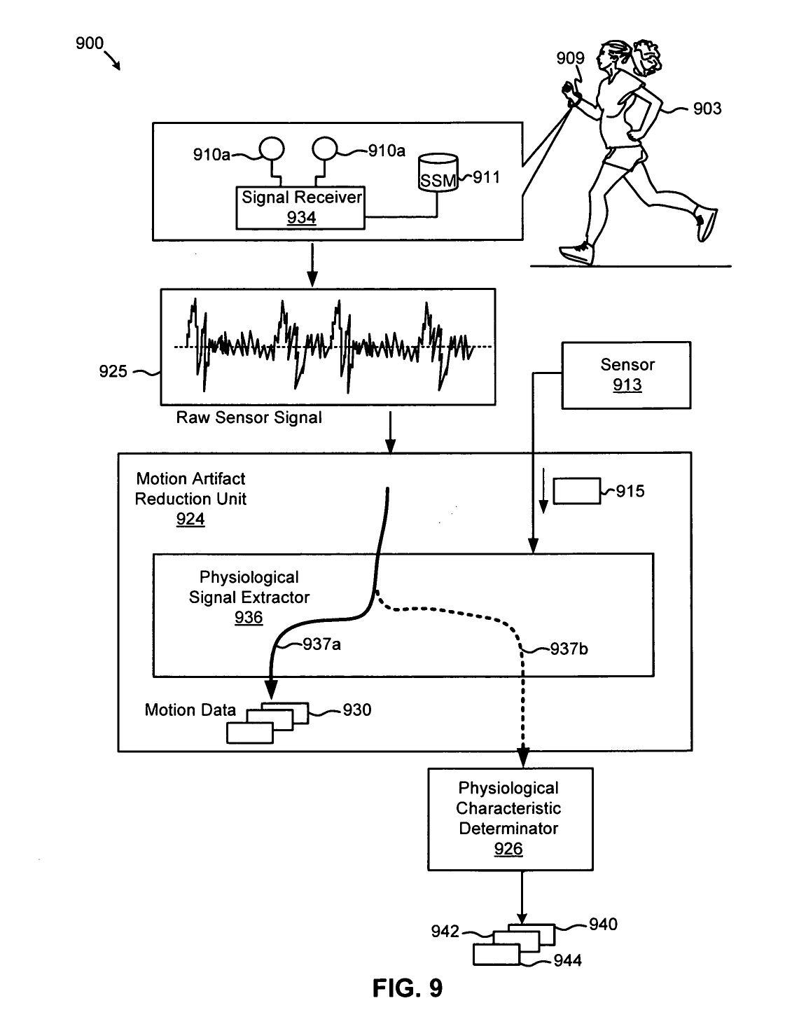

FIG. 10 is a flowchart for extracting a physiological signal, according to some embodiments. At 1002, a motion sensor signal is correlated to a sensor signal, which includes one or more physiological characteristic signals and one or more motion-related artifact signals. In some examples, correlating motion sensor signals to bioimpedance signals enables the two signals to be compared against each other, whereby motion-related artifacts can be subtracted from the bioimpedance signals to extract a physiological characteristic signal. In at least one embodiment, data correlation at 1002 can be performed to include scaling data that represents a motion sensor signal, whereby the scaling facilitates making values for the data representing sensor signal equivalent so that they can be compared against each other (e.g., to facilitate subtracting one signal from the other). At 1004, a sensor signal is decomposed to extract one or more physiological signals and one or more motion sensor signals, thereby separating physiological signals from the motion signals. The extracted physiological signal is analyzed at 1006. In some examples, the frequency of the extracted physiological signal is analyzed to identify a dominant frequency component or predominant frequency components. Also, such an analysis at 1006 can also determine power spectral densities of the physiological extract physiological signal. At 1008, the relevant components of the physiological signal can be identified, based on the determination of the predominant frequency components. At 1010, at least one physiological signal is generated, such as a heart rate signal, a respiration signal, or a Mayer wave signal. These signals each can be associated with one or more corresponding dominant frequency component that are used to form the one or more physiological signals.

FIG. 11 is a block diagram depicting an example of a physiological signal extractor, according to some embodiments. Diagram 1100 depicts a physiological signal extractor 1136 that includes a stream selector 1140, a data correlator 1142, an optional window validator 1143, a parameter estimator 1144, and a separation filter 1146.

Physiological signal extractor 1136 can also include an optional offset generator 1139 to be discussed later. As shown in FIG. 11, physiological signal extractor 1136 receives a raw sensor signal from, for example, a bioimpedance sensor, and also receives one or more motion sensor signals 1143 from a motion sensor 1141, which can include one or more accelerometers in some examples. Multiple data streams can represent accelerometer data in multiple axes. Stream selector 1140 is configured to receive, for example, multiple accelerometer signals specifying motion along one or more different axes. Further, stream selector 1140 is configured to select an accelerometer data stream having a greatest motion component (e.g., the greatest magnitude of acceleration for an axis). In some examples, stream selector 1140 is configured to select the axis of acceleration having the highest variability in motion, whereby that axis can be used to track motion or identify a general direction or plane of motion. Optionally, offset generator 1139 can receive a magnitude of the raw sensor signal to modify the dynamic range of an amplifier receiving the raw sensor signal prior to that signal entering data correlator 1142.

Data correlator 1142 is configured to receive the raw sensor signal and the selected stream of accelerometer data. Data correlator 1142 operates to correlate the sensor signal and the selected motion sensor signal. For example, data correlator 1142 can scale the magnitudes of the selected motion sensor signal to an equivalent range for the sensor signal. In some embodiments, data correlator 1142 can provide for the transformation of the signal data between the bioimpedance sensor signal space and the acceleration data space. Such a transformation can be optionally performed to make the motion sensor signals, especially the selected motion sensor signal, equivalent to the bioimpedance sensor signal. In some examples, a cross-correlation function or an autocorrelation function can be implemented to correlate the sets of data representing the motion sensor signal and the sensor signal.

Parameter estimator 1144 is configured to receive the selected motion sensor signal from stream selector 1140 and the correlated data signal from data correlator 1142. In some examples, parameter estimator 1144 is configured to estimate parameters, such as coefficients, for filtering out physiological characteristic signals from motion-related artifact signals. For example, the selected motion sensor signal, such as accelerometer signal, generally does not include biological derived signal data, and, as such, one or more coefficients for physiological signal components can be reduced or effectively determined to be zero. Separation filter 1146 is configured to receive the coefficients as well as data correlated by data correlator 1142 and the selected motion sensor signal from stream selector 1140. In operation, separation filter 1146 is configured to recover the sources of the signals. For example, separation filter 1146 can generate a recovered physiological characteristic signal (“P”) 1160 and a recovered motion signal (“M”) 1162. Separation filter 1146, therefore, operates to separate a sensor signal including both biological signals and motion-related artifact signals into additive or subtractable components. Recovered signals 1160 and 1162 can be used to further determine one or more physiological characteristics signals, such as a heart rate, respiration rate, and a Mayer wave.

Window validator 1143 is optional, according to some embodiments. Window validator 1143 is configured to receive motion sensor signal data to determine a duration time (i.e., a valid window of time) in which sensor signal data can be predicted to be valid (i.e., durations in which the magnitude of motion-related artifacts signals likely do not affect the physiological signals). In some cases, window validator 1143 is configured to predict a saturation condition for a front-end amplifier (or any other condition, such as a motion-induced condition), whereby the sensor signal data is deemed invalid.

FIG. 12 depicts an example of an offset generator according to some embodiments. Diagram 1200 depicts offset generator 1239 including a dynamic range determinator 1240 and an optional amplifier 1242, which can be disposed within or without offset generator 1239. In sensing bioimpedance-related signals, the bioimpedance signals generally are “small-signal;” that is, these signals have relatively small amplitudes that can be distorted by changes in impedances, such as when the coupling between the electrodes and the skin is disrupted. Offset generator 1239 can be configured to determine an amount of motion that is associated with motion sensor signal (“M”) 1260, such as an accelerometer signal, and to adjust the dynamic range of operation of amplifier 1242, which can be an operational amplifier configured as a front-end amplifier. Further, offset generate 1239 can also be optionally configured to receive sensor signal (“S”) 1262 and correlated data (“CD”) 1264, either or both of which can be used to determine first whether to modify the dynamic range of amplifier 1242, and if so, to what degree to which the dynamic range ought to be modified. In some cases, the degree to which the dynamic range ought to be modified specified by an offset value. As shown, amplifier 1242 is configured to generate an offset sensor signal that is conditioned or otherwise adapted to avoid or reduce clipping.

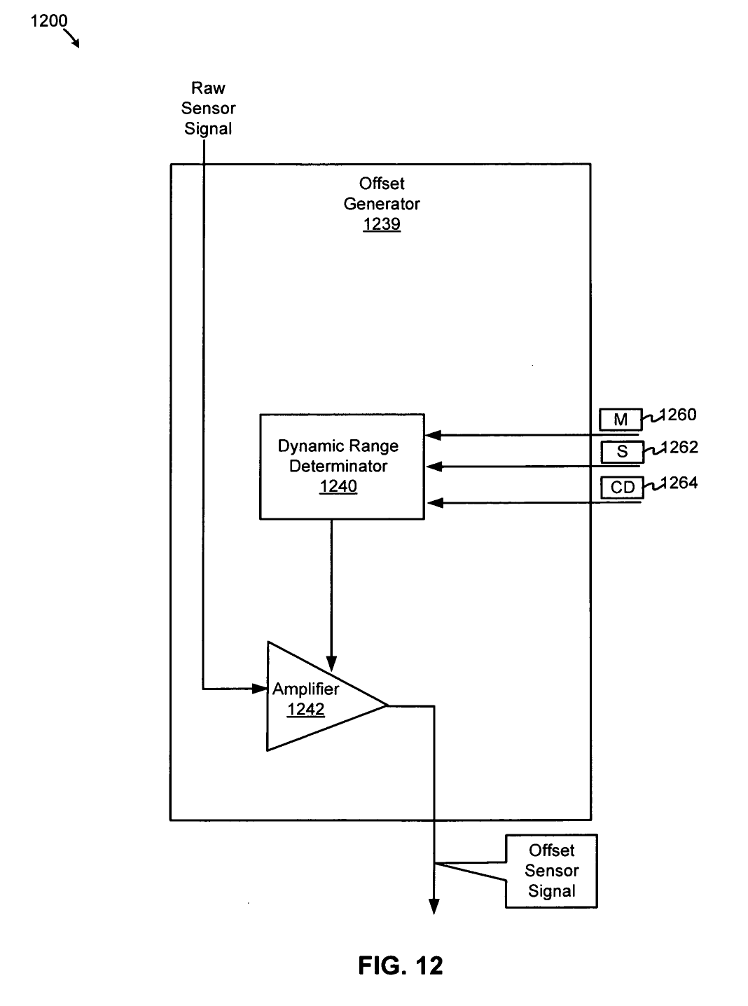

FIG. 13 is a flowchart depicting example of a flow for decomposing a sensor signal to form separate signals, according to some embodiments. Flow 1300 can be implemented in a variety of different ways using a number of different techniques. In some examples, flow 1300 and its elements can be implemented by one or more of the components or elements described herein, according to various embodiments. In the following example, while not intended to be limiting, flow 1300 is described in terms of an analysis for extracting physiological characteristic signals in accordance with one or more techniques of performing Independent Component Analysis (“ICA”). At 1302, a sensor signal is received, and at 1304 a motion sensor signal is selected. When a test subject, or user, is wearing a wearable device and is physically active, the received bioimpedance signal can include two signals: 1.) a sensor signal including one or more physiological signals such as heart rate, respiration rate, and Mayer waves, and 2.) motion-related artifact signals. Further, the one or more physiological signals and motion sensor signals (or motion-related artifact signals) may be correlated at 1305. In this example, a physiological signal is assumed to be statistically independent (or nearly statistically independent) of a motion sensor signal or related artifacts. In some examples, flow 1300 provides for separating a multivariate signal into additive or subtractive subcomponents, based on a presumed mutually-statistical independence between non-Gaussian source signals. Statistical independence of estimated physiological sample components and motion related artifact signal components can be maximized based on for example minimizing mutual information, and maximizing non-Gaussianity of the source signals.

Further to flow 1300, consider two statistically independent noun Gaussian source signals S1 and S2, and two observation points O1 and O2. In some examples, observation points O1(t) and O2(t) are time-indexed samples associated with observed samples from the same sensor, at different locations. For example, O1(t) and O2(t) can represent observed samples from a first bioimpedance sensor (or electrode) and from a second bioimpedance sensor (or electrode), respectively. In other examples, O1(t) and O2(t) can represent observed samples from a first sensor, such as a bioimpedance sensor, and a second sensor, such as an accelerometer, respectively. At 1306, data associated with one or more of the two observation points O1 and O2 are preprocessed. For example, the data for the observation points can be centered, whitened, and/or reduced in dimensions, wherein preprocessing may reduce the complexity of determining the source signals and/or reduce the number of parameters or coefficients to be estimated. An example of a centering process includes subtracting the meaning of data from a sample to translate samples about a center. An example of a whitening process is eigenvalue decomposition. In some embodiments, preprocessing at 1306 can be different from, or similar to, the correlation of data as described herein, at least in some cases.

Observation points O1(t) and O2(t) can be expressed as follows:

O₁(t)=a₁₁S1+a₁₂S2 (Eqn. 1)

O₂(t)=a₂₁S1+a₂₂S2 (Eqn. 2)

where O=A×S, which represent matrices, and a11, a12, a21, and a22 represent parameters (or coefficients) that can be estimated. At 1308, the above equations 1 and 2 can be used to determine components for generating two (2) statistically-independent source signals, whereby A and S can be extracted from O. In some examples, A and S can be extracted iteratively, based on user-specified error rate and/or maximum number of iterations, among other things. Further, coefficients a11, a12, a21, and a22 can be modified such that one or more coefficients for the physiological characteristic and biological component is set to or near zero, as the accelerometer signal generally does not include physiological signals. In at least one embodiment, parameter estimator 1144 of FIG. 11 can be configured to determine estimated coefficients.

In some examples a matrix can be formed based on estimated coefficients, at 1308. At least some of the coefficients are configured to attenuate values of the physiological signal components for the motion sensor signal. An example of the matrix is a mixing matrix. Further, the matrix of coefficients can be inverted to form an inverted mixing matrix (e.g., to form an “unmixing” matrix). The inverted mixing matrix of coefficients can be applied (e.g., iteratively) to the samples of observation points O1(t) and O2(t) to recover the source signals, such as a recovered physiological characteristic signal and a recovered motion signal (e.g. a recovered motion-related artifact signal). In at least one embodiment, separation filter 1146 of FIG. 11 can be configured to apply an inverted matrix to samples of the physiological signal components and the motion signal components to determine the recovered physiological characteristic signal and the recovered motion signal (e.g., a recovered muscle movement signal). Note that various described functionalities of flow 1300 can be implemented in or distributed over one or more of the described structures set forth herein. Note, too, that while flow 1300 is described in terms of ICA in the above-mentioned examples, flow 1300 can be implemented using various techniques and structures, and the various embodiments are neither restricted nor limited to the use of ICA. Other signal separation processes may also be implemented, according to various embodiments.

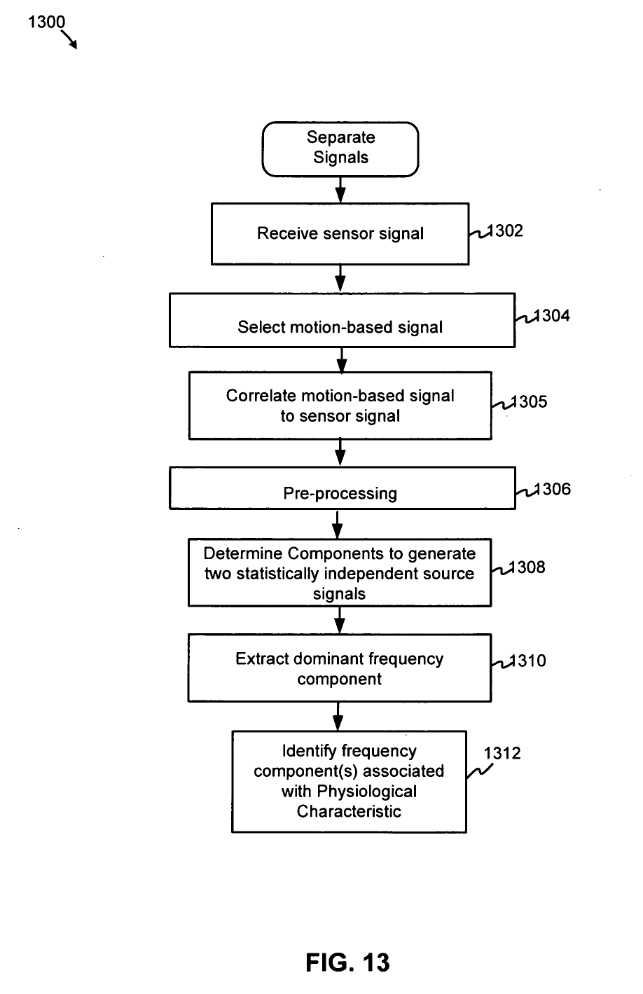

FIGS. 14A to 14D depict various signals used for physiological characteristic signal extraction, according to various embodiments. FIG. 14A depicts a sensor signal received as, for example, a bioimpedance signal in which the magnitude varies about 20 over a number of samples. In this example, validation window can be used for heart rate extraction, whereby the sensor signal is down-sampled by, for example, a factor of 100 (i.e., the sensor signal is sampled at, for example, 15.63 Hz). Also shown in FIG. 14A is an optional window 1402 that indicates a validation window in which data is deemed valid as determined by, for example, window validator 1143 of FIG. 11. Returning back to FIGS. 14A to 14D, FIG. 14B depicts a first stream of accelerometer data for a first axis. FIG. 14C and FIG. 14D depict a second stream of accelerometer data for a second axis and a third stream of accelerometer data for a third axis, respectively. FIGS. 14A to 14D are intended to depict only a few of many examples and implementations.

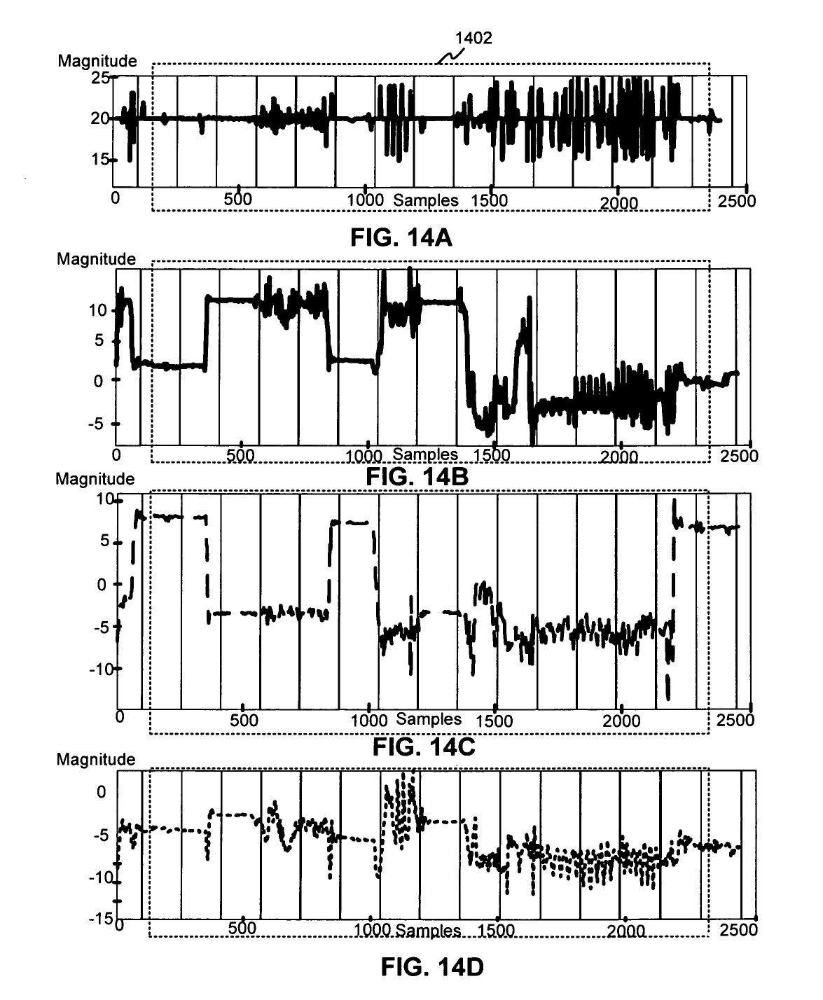

FIG. 15 depicts recovered signals, according to some embodiments. Diagram 1500 depicts the magnitudes of various signals over 160 samples. Signal 1502 represents us magnitude of the sensor signal, whereas signal 1504 represents the magnitude of an accelerometer signal. Signals 1506, 1508, and 1510 represent the magnitudes of a first of accelerometer signal, a second accelerometer signal, and a third accelerometer signal, respectively.

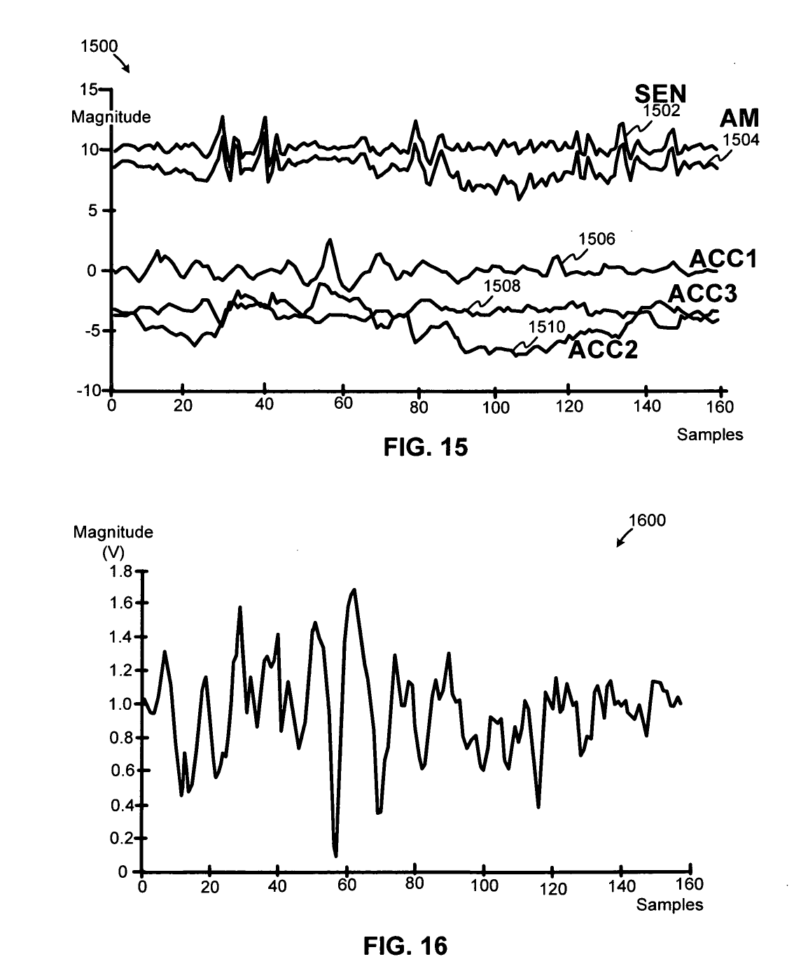

FIG. 16 depicts an extracted physiological signal, according to various embodiments. Diagram 1600 depicts the magnitude, in volts, of an extracted physiological characteristic signal using the first accelerometer stream as the selected accelerometer stream. For this example, a fast Fourier transform (“FFT”) analysis of the data set forth in FIG. 16 yields a heart rate estimated at, for example, 77.6274 bpm.

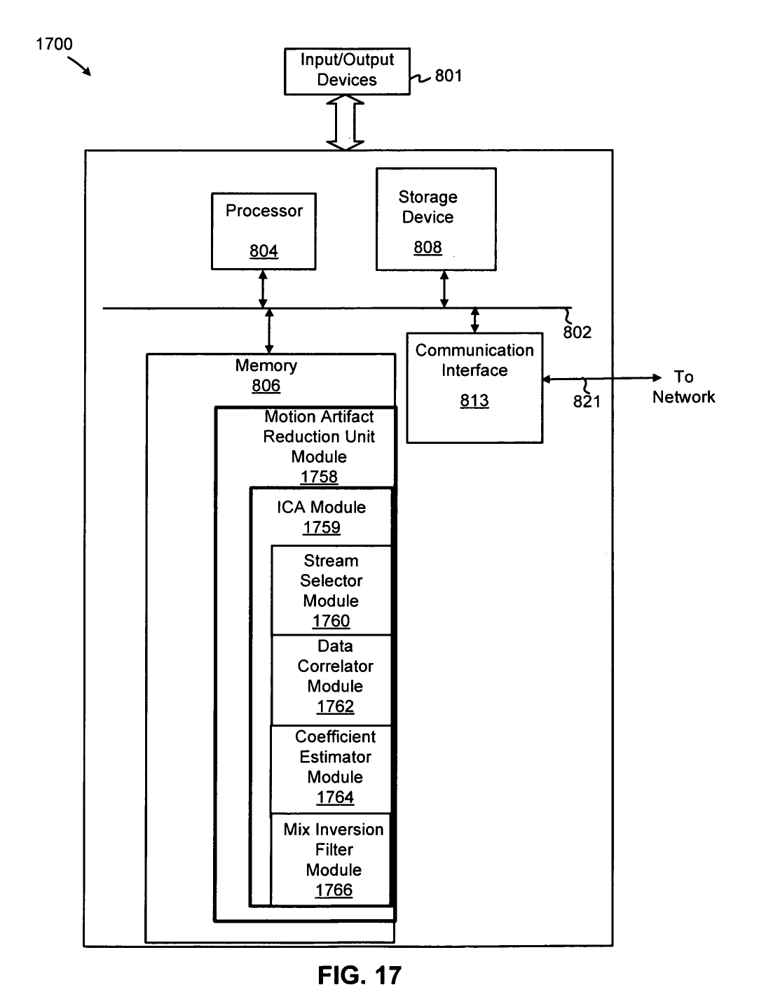

FIG. 17 illustrates an exemplary computing platform disposed in a wearable device in accordance with various embodiments. In some examples, computing platform 1700 may be used to implement computer programs, applications, methods, processes, algorithms, or other software to perform the above-described techniques, and can include similar structures and/or functions as set forth in FIG. 8. But in the example shown, system memory 806 can include various modules that include executable instructions to implement functionalities described herein. In the example shown, system memory 806 includes a motion artifact reduction unit module 1758 configured to determine physiological information relating to a user that is wearing a wearable device. Motion artifact reduction unit module 1758 can include a stream selector module 1760, a data correlator module 1762, a coefficient estimator module 1764, and a mix inversion filter module 1766, any of which can be configured to provide one or more functions described herein.

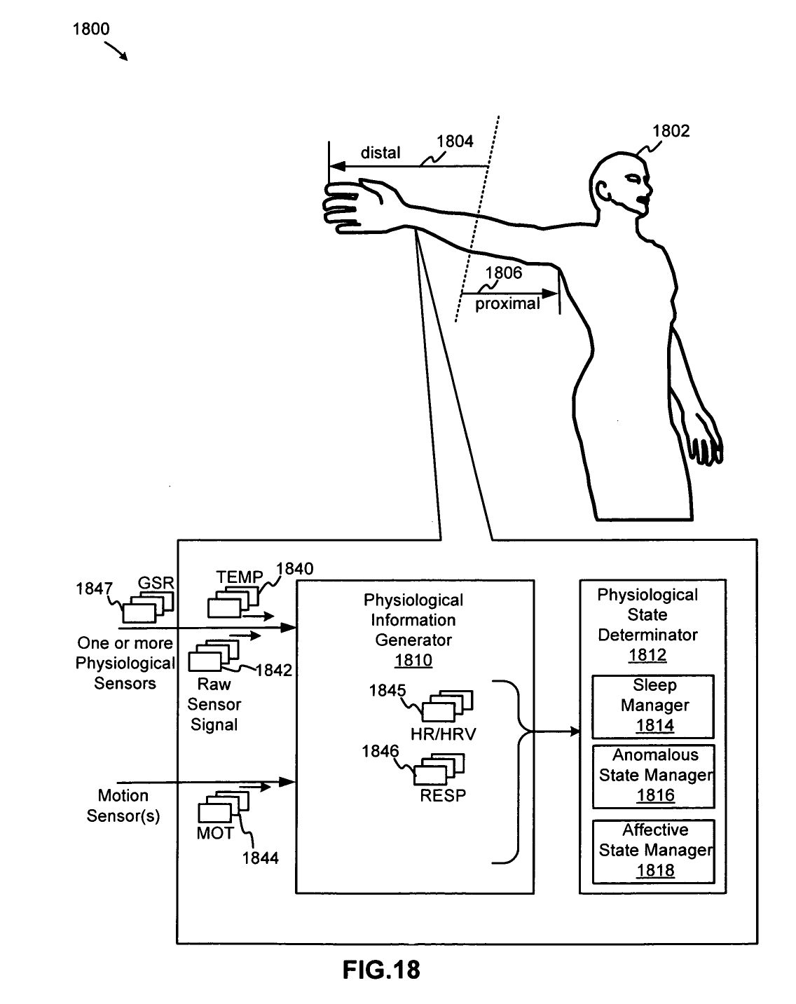

FIG. 18 is a diagram depicting a physiological state determinator configured to receive sensor data originating, for example, at a distal portion of a limb, according to some embodiments. As shown, diagram 1800 depicts a physiological information generator 1810 and a physiological state determinator 1812, which, at least in the example shown, are configured to be disposed at, or receive signals from, at a distal portion 1804 of a user 1802. In some embodiments, physiological information generating 1810 and physiological state determinator 1812 are disposed in a wearable device (not shown). Physiological information generator 1810 configured to receive signals and/or data from one or more physiological sensors and one or more motion sensors, among other types of sensors. In the example shown, physiological information generator 1810 is configured to receive a raw sensor signal 1842, which can be similar or substantially similar to other raw sensor signals described herein. Physiological information generator 1810 is also configured to receive other sensor signals including temperature (“TEMP”) 1840, skin conductance (depicted as GSR data signal 1847), pulse waves, heat rates (e.g., heart beats-per-minute), respiration rates, heart rate variability, and any other sensed signal configured to include physiological information or any other information relating to the physiology of a person. Examples of other sensors are described in U.S. patent application Ser. No. 13/454,040, filed on Apr. 23, 2012, which is incorporated by reference. Physiological information generator 1810 is also configured to receive motion (“MOT”) signal data 1844 from one or more motion sensor(s), such as accelerometers. Note that raw sensor signal 1842 can be an electrical signal, such as a bioimpedance signal, or an acoustic signal, or any other type of signal. According to some embodiments, physiological information generator 1810 is configured to extract physiological signals from a raw sensor signal 1842. For example, a heart rate (“HR”) signal and/or heart rate variability (“HRV”) signal 1845 and respiration rate (“RESP”) 1846 can be determined for example, by a motion artifact reduction unit (not shown). Physiological information generator 1810 is configured to convey sensed physiological characteristics signals or derive physiological characteristic signals (e.g., from sensed signals) for use by physiological state determinator 1812. In some examples, a physiological characteristic signal can include electrical impulses of muscles (e.g., as evidenced, in some cases, by electromyography (“EMG”) to determine the existence and/or amounts of motion based on electrical signals generated by muscle cells at rest or in contraction.

As shown, physiological state determinator 1812 includes a sleep manager 1814, an anomalous state manager 1816, and an affective state manager 1818. Physiological state determinator 1812 is configured to receive various physiological characteristics signals and to determine a physiological state of a user, such as user 1802. Physiological states include, but are not limited to, states of sleep, wakefulness, a deviation from a normative physiological state (i.e., an anomalous state), an affective state (i.e., mood, feeling, emotion, etc.). Sleep manager 1814 is configured to detect a stage of sleep as a physiological state, the stages of sleep including REM sleep and non-REM sleep, including as light sleep and deep sleep. Sleep manager 1814 is also configured to predict the onset or change into or between different stages of sleep, even if such changes are imperceptible to user 1802. Sleep manager 1814 can detect that user 1802 is transitioning from a wakefulness state to a sleep state and, for example, can generate a vibratory response (i.e., generated by vibration) or any other alert to user 1802. Sleep manager 1814 also can predict a sleep stage transition to either alert user 1802 or to disable such an alert if, for example, the alert is an alarm (i.e., wake-up time alarm) that coincides with a state of REM sleep. By delaying generation of an alarm, the user 1802 is permitted to complete of a state of REM sleep to ensure or enhance the quality of sleep. Such an alert can assist user 1802 to avoid entering a sleep state from a wakefulness state during critical activities, such as driving.

Anomalous state manager 1860 is configured to detect a deviation from the normative general physiological state in reaction, for example, to various stimuli, such as stressful situations, injuries, ailments, conditions, maladies, manifestations of an illness, and the like. Anomalous state manager 1860 can be configured to determine the presence of a tremor that, for example, can be a manifestation of an ailment or malady. Such a tremor can be indicative of a diabetic tremor, an epileptic tremor, a tremor due to Parkinson's disease, or the like. In some embodiments, anomalous state manager 1860 is configured to detect the onset of tremor related to a malady or condition prior to user 1802 perceiving or otherwise being aware of such a tremor. Therefore, anomalous state manager 1860 can predict the onset of a condition that may be remedied by, for example, medication and can alert user 1802 to the impending tremor. User 1802 then can take the medication before the intensity of the tremor increases (e.g., to an intensity that might impair or otherwise incapacitate user 1802). Further, anomalous state manager 1860 can be configured to determine if the physiological state of user 1802 is a pain state, in which user 1802 is experiencing pain. Upon determining a pain state, a wearable device (not shown) can be configured to transmit the presence of pain to a third-party via a wireless communication path to alert others of the pain state for resolution.

Affective state manager 1818 is configured to use at least physiological sensor data to form affective state data representing an approximate affective state of user 1802. As used herein, the term “affective state” can refer, at least in some embodiments, to a feeling, a mood, and/or an emotional state of a user. In some cases, affective state data can includes data that predicts an emotion of user 1802 or an estimated or approximated emotion or feeling of user 1802 concurrent with and/or in response to the interaction with another person, environmental factors, situational factors, and the like. In some embodiments, affective state manager 1818 is configured to determine a level of intensity based on sensor derived values and to determine whether the level of intensity is associated with a negative affectivity (e.g., a bad mood) or positive affectivity (e.g., a good mood). An example of an affective state manager 1818 is an affective state prediction unit as described in U.S. Provisional Patent Application No. 61/705,598 filed on Sep. 25, 2012, which is incorporated by reference herein for all purposes. While affective state manager 1818 is configured to receive any number of physiological characteristics signals in which to determine of an affective state of user 1802, affective state manager 1818 can use sensed and/or derived Mayer waves based on raw sensor signal 1842. In some examples, the detected Mayer waves can be used to determine heart rate variability (“HRV”) as heart rate variability can be correlated to Mayer waves. Further, affective state manager 1818 can use, at least in some embodiments, HRV to determine an affective state or emotional state of user 1802 as HRV may correlate with an emotion state of user 1802. Note that, while physiological information generating 1810 and physiological state determinator 1812 are described above in reference to distal portion 1804, one or more of these elements can be disposed at, or receive signals from, proximal portion 1806, according to some embodiments.

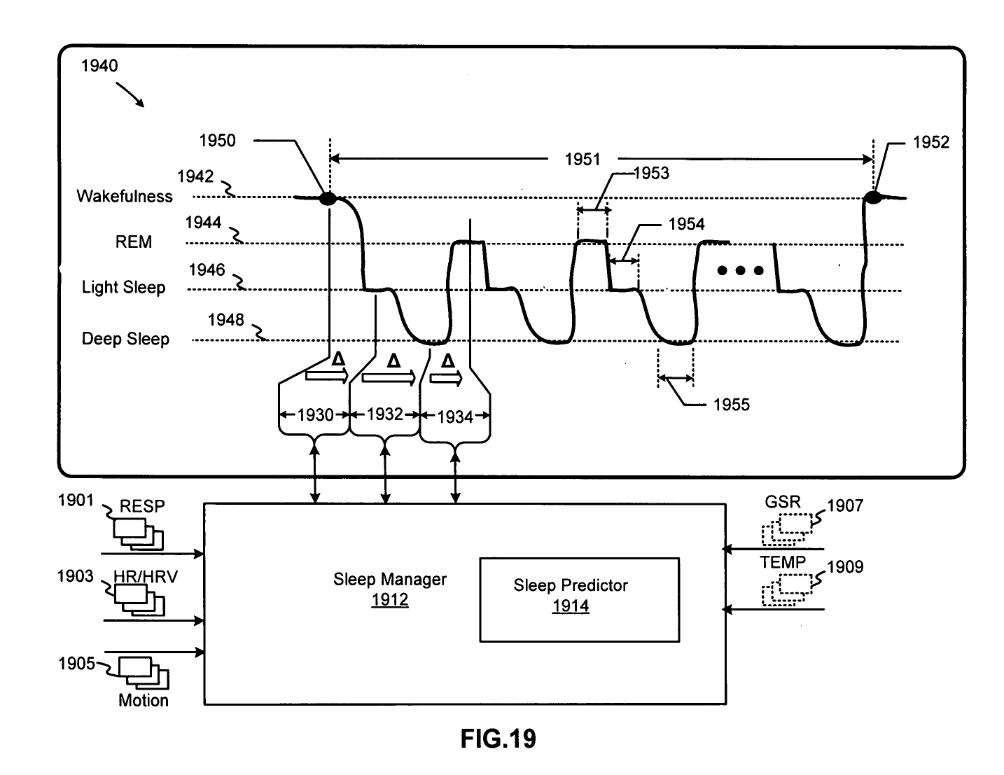

FIG. 19 depicts a sleep manager, according to some embodiments. As shown, FIG. 19 depicts a sleep manager 912 including a sleep predictor 1914. Sleep manager 1912 is configured to determine physiological states of sleep, such as a sleep state or a wakefulness state in which the user is awake. Sleep manager 1912 is configured to receive physiological characteristic signals, such as data representing respiration rates (“RESP”) 1901, heart rate (“HR”) 1903 (or heart rate variability, HRV), motion-related data 1905, and other physiological data such as optional skin conductance (“GSR”) 1907 and optional temperature (“TEMP”) 1909, among others. As shown in diagram 1940, a person who is sleeping passes through one or more sleep cycles over a duration 1951 between a sleep start time 1950 and sleep end time 1952. There is a general reduction of motion when a person passes from a wakefulness state 1942 into the stages of sleep, such as into light sleep 1946 in duration 1954. Motion indicative of “hypnic jerks” or involuntary muscle twitching motions typically occur during light sleep state 1946. The person then passes into a deep sleep state 1948, in which, a person has a decreased heart rate and body temperature, with the absence of voluntary muscle motions to confirm or establish that a user is in a deep sleep state. Collectively, the light sleep state and the deep sleep state can be described as non-REM sleep states. Further to diagram 1940, the sleeping person then passes into an REM sleep state 1944 for duration 1953 during which muscles can be immobile.

According to some embodiments, sleep manager 1912 is configured to determine a stage of sleep based on at least the heart rate and respiration rate. For example, sleep manager 1912 can determine the regularity of the heart rate and respiration rate to determine the person is in a non-REM sleep state, and, thereby, can generate a signal indicating the stage of the sleep is a non-REM sleep states, such as light sleep or deep sleep states. During light sleep and deep sleep, a heart rate and/or the respiration rate of the user can be described as regular or without significant variability. Thus, the regularity of the heart rate and/or respiration rate can be used to determine physiological sleep state of the user. In some examples the regularity of the heart rate and/or the respiration rate can include any heart rate or respiration rate that varies by no more than 5%. In some other cases, the regularity of the heart rate and/or the respiration rate can vary by any amount up to 15%. These percentages are merely examples and are not intended to be limiting, and ordinarily skilled artisan will appreciate that the tolerances for regular heart rates and respiration rates may be based on user characteristics, such as age, level of fitness, gender and the like. Sleep manager 1912 can use motion data 1905 to confirm whether a user is in a light sleep state or a deep sleep state by detecting indicative amounts of motion, such as a portion of motion that is indicative of involuntary muscle twitching.

As another example, sleep manager 1912 can determine the irregularity (or variability) of the heart rate and respiration rate to determine the person is in an REM sleep state, and, thereby, can generate a signal indicating the stage of the sleep is an REM sleep states. During REM sleep, a heart rate and/or the respiration rate of the user can be described as irregular or with sufficient variability to identify that a user is REM sleep.

Thus, the variability of the heart rate and/or respiration rate can be used to determine physiological sleep state of the user. In some examples the irregularity of the heart rate and/or the respiration rate can include any heart rate or respiration rate that varies by more than 5%. In some other cases, the variability of the heart rate and/or the respiration rate can vary by any amounts up from 10% to 15%. These percentages are merely examples and are not intended to be limiting, and ordinarily skilled artisan will appreciate that the tolerances for variable heart rates and respiration rates may be based on user characteristics, such as age, level fitness, gender and the like. Sleep manager 1912 can use motion data 1905 to confirm whether a user is in an REM sleep state by detecting indicative amounts of motion, such as a portion of motion that includes negligible to no motion.

Sleep manager 1912 is shown to include sleep predictor 1914, which is configured to predict the onset or change into or between different stages of sleep. The user may not perceive such changes between sleep states, such as transitioning from a wakefulness state to a sleep state. Sleep predictor 1914 can detect this transition from a wakefulness state to a sleep state, as depicted as transition 1930. Transition 1930 may be determined by sleep predictor 1940 based on the transitions from irregular heart rate and respiration rates during wakefulness to more regular heart rates and respiration rates during early sleep stages. Also, lowered amounts of motion can also indicate transition 1930. In some embodiments, motion data 1905 includes a velocity or rate of speed at which a user is traveling, such as an automobile. Upon detecting an impending transition from a wakefulness state into a sleep state, sleep predictor 1914 generates an alert signal, such as a vibratory initiation signal, configuring to generate a vibration (or any other response) to convey to a user that he or she is about to fall asleep. So if the user is driving, predictor 914 assists in maintaining a wakefulness state during which the user can avoid falling asleep behind the wheel. Sleep predictor 1914 can be configured to also detect transition 1932 from a light sleep state to a deep sleep state and a transition 1934 from a deep sleep state to an REM sleep state. In some embodiments, transitions 1932 in 1934 can be determined by detected changes from regular to variable heart rates or respiration rates, in the case of transition 1934. Also, transition 1934 can be described by a decreased level of motion to about zero during the REM sleep state. Further, sleep predictor 1914 can be configured to predict a sleep stage transition to disable an alert, such as wake-up time alarm, that coincides with a state of REM sleep. By delaying generation of an alarm, the user is permitted to complete of a state of REM sleep to enhance the quality of sleep. In some embodiments, sleep manager 1912 detects increase perspiration via skin conductance during an REM sleep state and determines the user is dreaming, whereby in generates a signal to store such an event or generate an other action.

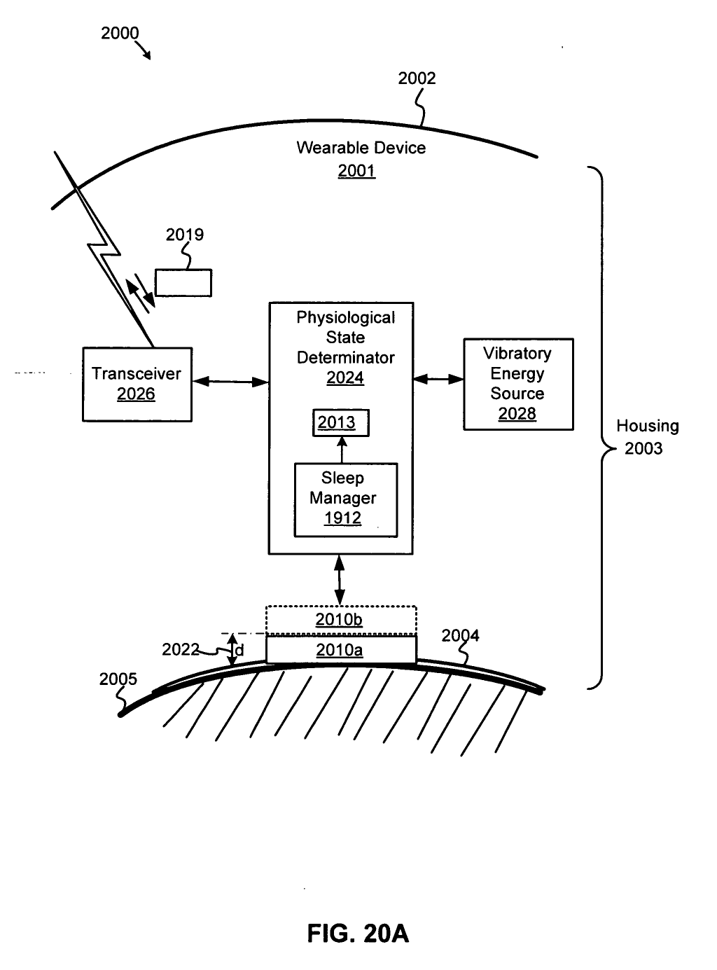

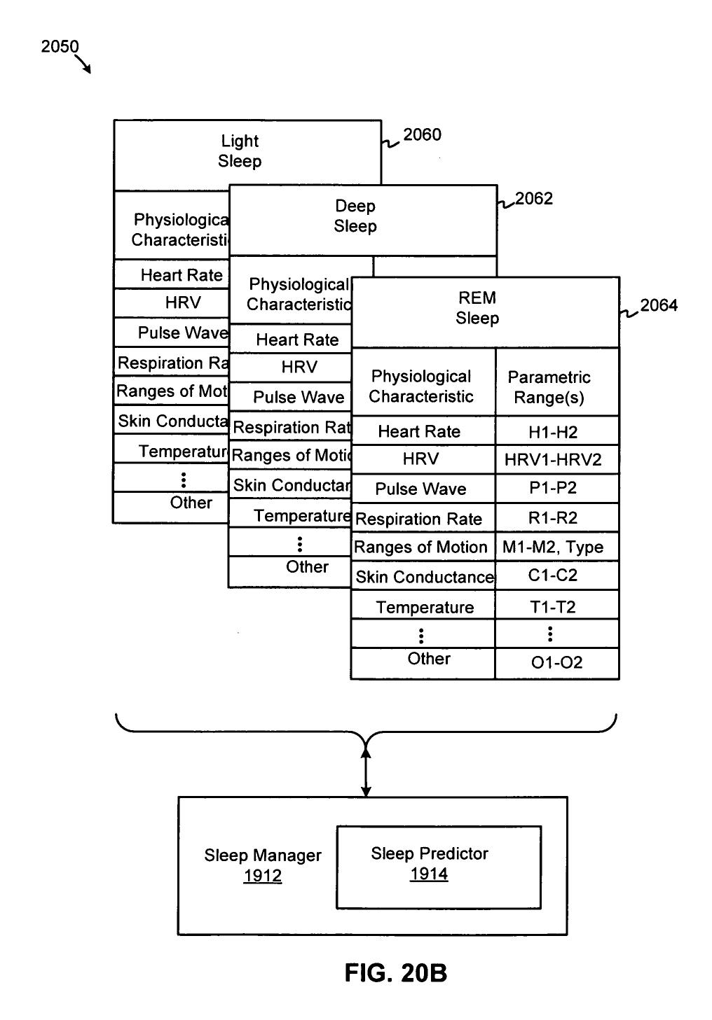

FIG. 20A depicts a wearable device including a skin surface microphone (“SSM”), in various configurations, according to some embodiments. According to various embodiments, a skin surface microphone (“SSM”) can be implemented in cooperation with (or along with) one or more electrodes for bioimpedance sensors, as described herein. In some cases, a skin surface microphone (“SSM”) can be implemented in lieu of electrodes for bioimpedance sensors. Diagram 2000 of FIG. 20 depicts a wearable device 2001, which has an outer surface 2002 and an inner surface 2004. In some embodiments, wearable device 2001 includes a housing 2003 configured to position a sensor 2010a (e.g., an SSM including, for instance, a piezoelectric sensor or any other suitable sensor) to receive an acoustic signal originating from human tissue, such as skin surface 2005. As shown, at least a portion of sensor 2010a can be formed external to surface 2004 of wearable housing 2003. The exposed portion of the sensor can be configured to contact skin 2005. In some embodiments, the sensor (e.g., SSM) can be disposed at position 2010b at a distance (“d”) 2022 from inner surface 2004. Material, such as an encapsulant, can be used to form wearable housing 2003 to reduce or eliminate exposure to elements in the environment external to wearable device 2001. In some embodiments, a portion of an encapsulant or any other material can be disposed or otherwise formed at region 2010a to facilitate propagation of an acoustic signal to the piezoelectric sensor. The material and/or encapsulant can have an acoustic impedance value that matches or substantially matches the acoustic impedance of human tissue and/or skin. Values of acoustic impedance of the material and/or encapsulant can be described as being substantially similar to the human tissue and/or skin when the acoustic impedance of the material and/or encapsulant varies no more than 60% of that of human tissue or skin, according to some examples.

Examples of materials having acoustic impedances matching or substantially matching the impedance of human tissue can have acoustic impedance values in a range that includes 1.5×106 Pa×s/m (e.g., an approximate acoustic impedance of skin) In some examples, materials having acoustic impedances matching or substantially matching the impedance of human tissue can provide for a range between 1.0×106 Pa×s/m and 1.0×107 Pa×s/m. Note that other values of acoustic impedance can be implemented to form one or portions of housing 2003. In some examples, the material and/or encapsulant can be formed to include at least one of silicone gel, dielectric gel, thermoplastic elastomers (TPE), and rubber compounds, but is not so limited. As an example, the housing can be formed using Kraiburg TPE products. As another example, housing can be formed using Sylgard® Silicone products. Other materials can also be used.

Further to FIG. 20A, wearable device 2001 also includes a physiological state determinator 2024, a sleep manager 1912, a vibratory energy source 2028, and a transceiver 2026. Physiological state determinator 2024 can be configured to receive signals originating as acoustic signals either from sensor 2010a or a sensor at location 2010b via acoustic impedance-matched material. Upon detecting a sleep state condition (e.g., a sleep state transition), sleep manager 1912 can be configured to communicate the condition to physiological state determinator 2024, which, in turn, generates a notification signal as a vibratory activation signal, thereby causing vibratory energy source 2028 (e.g., mechanical motor as a vibrator) to impart vibration through housing 2003 unto a user, responsive to the vibratory activation signal, to indicate the presence of the sleep-related condition (e.g., transitioning from a wakefulness state to a sleep state).

According to some embodiments, sleep manager 1912 can generate a wake enable/disable signal 2013 configured to enable or disable the ability of vibratory energy source 2028 to generate an alarm signal. For example, if sleep manager 1912 determines that the user is in a REM sleep state, sleep manager 1912 generates a wake disable signal 2013 to prevent vibratory energy source 2228 from waking the user. But if sleep manager 1912 determines that the user is in a non-REM sleep state that coincides with a wake alarm time, or is there shortly thereafter, sleep manager 1912 will generate enable signal 2013 to permit vibratory energy source 2028 to wake up the user. In some cases, a wake enable signal and awake disable signal can be the same signal, but at different states. Also, wearable device 2001 can optionally include a transceiver 2026 configured to transmit signal 2019 as a notification signal via, for example, an RF communication signal path. In some examples, transceiver 2026 can be configured to transmit signal 2019 to include data representative of the acoustic signal received from sensor 2010, such as an SSM.

FIG. 20B depicts an example of physiological characteristics and parametric values that can identify a sleep state, according to some embodiments. Diagram 2050 depicts a data arrangement 2060 including data for determining light sleep states, a data arrangement 2062 that includes data for determining deep sleep states, and data arrangement 2064 that includes data for determining REM sleep states, according to various embodiments. Also shown in FIG. 20B, sleep manager 1912 and sleep predictor 1914 can use data arrangements 2060, 2062 and 2064 to determine the various sleep stages of the user. As shown generally, each of the sleep states can be defined one or more physiological characteristics, such as heart rate, HRV, pulse wave, respiration rate, ranges of motion, types of motion, skin conductance, temperature, and any other physiological characteristic or information. As shown, each physiological characteristic is associated with a parametric range that may include one or more than one value associated with the physical physiological characteristic. For example, should the heart rate of a user fall within the range H1-H2, as shown in data arrangement 2064, sleep manager can use this information in determining whether the user is in REM sleep. In some cases, the parametric values that set forth the ranges, maybe based on characteristics of a user, such as age, level of fitness, gender, etc. In one example, sleep manager 1912 operates to analyze the various values of the physiological characteristics and calculates a best-fit determination of the parametric values to identify the corresponding sleep state for the user. The physiological characteristics and parametric values, and data arrangements 2062 to 2064 is merely one example and is not intended to be limiting.

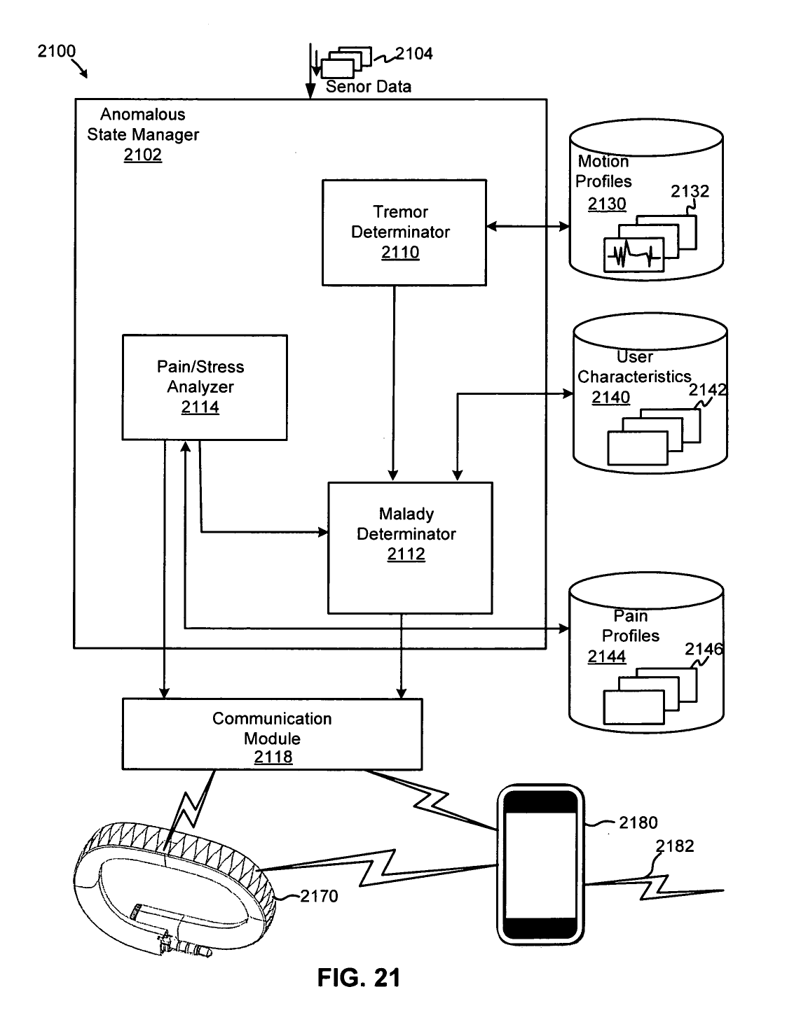

FIG. 21 depicts an anomalous state manager 2102, according to some embodiments. Diagram 2100 depicts that anomalous state manager 2102 includes a tremor determinator 2110, a pain/stress analyzer 2114 and a malady determinator 2112. Anomalous state manager 2102 receives sensor data 2104 and is configured to detect a deviation from the normative general physiological state of a user responsive, for example, to various stimuli, such as stressful situations, injuries, ailments, conditions, maladies, manifestations of an illness, symptoms of a condition, and the like. Also shown in diagram 2100 are repositories accessible by anomalous state manager 2102, including motion profile repository 2130, user characteristic repository 2140 and pain profile repository 2144. Motion profile repository 2130 includes profile data 2132 that includes data defining configured to define a tremor, or a portion thereof, associated with detected motion. User characteristic repository 2140 includes user-related data 2142 that describes the user, for example, in terms of age, fitness level, gender, diseases, conditions, ailments, maladies, and any other characteristic that may influence the determination of the physiological state of the user. Pain profiles 2144 includes data 2146 that can define whether the user is in a pain state. In some embodiments, data 2146 is a data arrangement that includes physiological characteristics similar to those shown in FIG. 20B. For example, physiological signs of pain may include, for example, an increase in respiration rate, an increase in the length of a respiration cycle (e.g., deeper inhalation and exhalation), changes and/or variations in blood pressure, changes and/or variations in heart rate, an increase in perspiration (e.g., increased skin conductance), an increase in muscle tone (e.g., as determined by physiological characteristics indicating increased electrical impulses to or by musculature, and the like). Based on such physiological characteristics, pain/stress analyzer 2114 can be configured to detect that the user is experiencing pain, and in some cases, the level of pain. Further, pain/stress analyzer 2114 can be configured to transmit data representing pain state information to a communication module 2118 for transmitting of the pain state-related information via wearable device 2170 or other mobile devices 2180 to a third-party (or any other entity or computing device) via communications path 2182 (e.g., wireless communications path and/or networks).

Tremor determinator 2110 is configured to determine the presence of a tremor that, for example, can be a manifestation of an ailment or malady. As discussed, such a tremor can be indicative of a diabetic tremor, an epileptic tremor, a tremor due to Parkinson's disease, or the like. In some embodiments, tremor determinator 2110 is configured to detect the onset of tremor related to a malady or condition prior to a user perceiving or otherwise being aware of such a tremor. In particular, wearable devices disposed at a distal portion of a limb may be more likely, at least in some cases, to detect tremors more readily than when disposed at a proximal portion.

Therefore, anomalous state manager 2102 can predict the onset of a condition that may be remedied by, for example, medication and can alert a user to the impending tremor. In some cases, malady determinator 2112 is configured to receive data representing a tremor and data 2142 representing user characteristics, and is further configured to determine the malady afflicting the user. For example, if data 2142 indicates the user is a diabetic, the tremor data received from tremor determinator 2110 is likely to indicate a diabetic-related tremor. Therefore, malady determinator 2112 can be configured to generate an alert that, for example, the user's blood glucose is decreasing to low level amounts that cause such diabetic tremors. The alert can be configured to prompt the user to obtaining medication to treat the impending anomalous physiological state of the user. In another example, tremor determinator 2110 in malady determinator 2112 cooperate to determine that the user is experiencing and an epileptic tremor, and generates an alert to enable the user to either take medication or stop engaging in a critical activity, such as driving, before the tremors become worse (i.e., to an intensity that might impair or otherwise incapacitate the user). Upon detection of tremor and the corresponding malady, anomalous state manager 2102 transmits data indicating the presence of such tremors via communication module 2118 to wearable device 2170 or mobile computing device 2180, which, in turn, transmit via networks 2182 to a third-party or any other entity. In some examples, anomalous state manager 2102 is configured to distinguish malady-related tremors from movements and/or shaking due to nervousness and or injury.

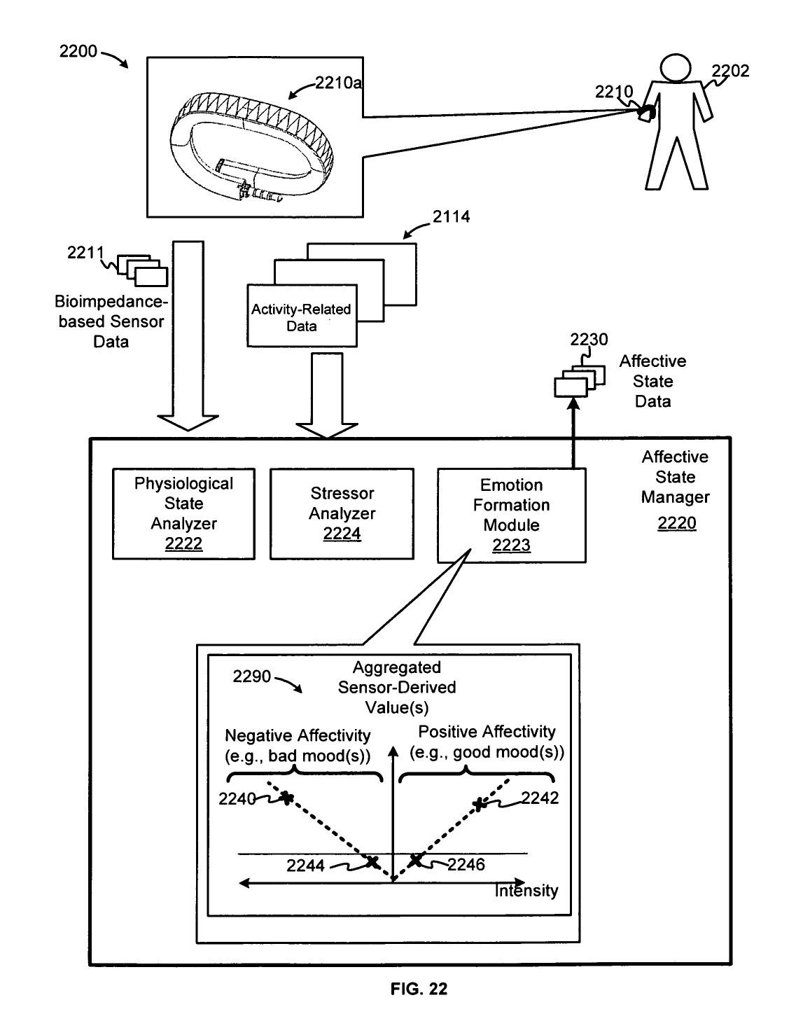

FIG. 22 depicts an affective state manager configured to receive sensor data derived from bioimpedance signals, according to some embodiments. FIG. 22 illustrates an exemplary affective state manager 2220 for assessing affective states of a user based on data derived from, for example, a wearable computing device, according to some embodiments. Diagram 2200 depicts a user 2202 including a wearable device 2210, whereby user 2202 experiences one or more types of stimuli that can changes in physiological states of user 2202, such as the emotional state of mind. In some embodiments, wearable device 2210 is a wearable computing device 2210a that includes one or more sensors to detect attributes of the user, the environment, and other aspects of the responses from/interaction with stimuli.

Affective state manager 2220 is shown to include a physiological state analyzer 2222, a stressor analyzer 2224, and an emotion formation module 2223. According to some embodiments, physiological state analyzer 2222 is configured to receive and analyze the sensor data, such as bioimpedance-based sensor data 2211, to compute a sensor-derived value representative of an intensity of an affective state of user 2202. In some embodiments, the sensor-derived value can represent an aggregated value of sensor data (e.g., an aggregated an aggregated value of sensor data value). In some examples, aggregated value of sensor data can be derived by, first, assigning a weighting to each of the values (e.g., parametric values) sensed by the sensors associated with one or more physiological characteristics, such as those shown in FIG. 20B, and, second, aggregating each of the weightings to form an aggregated value. Affective state manager 2220 can also receive activity-related data 2114 from a number of activity-related managers (not shown). One or more activity-related managers (not shown) can be configured to receive data representing parameters relating to one or more motion or movement-related activities of a user and to maintain data representing one or more activity profiles. Activity-related parameters describe characteristics, factors or attributes of motion or movements in which a user is engaged, and can be established from sensor data or derived based on computations. Examples of parameters include motion actions, such as a step, stride, swim stroke, rowing stroke, bike pedal stroke, and the like, depending on the activity in which a user is participating. As used herein, a motion action is a unit of motion (e.g., a substantially repetitive motion) indicative of either a single activity or a subset of activities and can be detected, for example, with one or more accelerometers and/or logic configured to determine an activity composed of specific motion actions.

According to some examples, the activity-related managers can include a nutrition manager, a sleep manager, an activity manager, a sedentary activity manager, and the like, examples of which can be found in U.S. patent application Ser. No. 13/433,204, filed on Mar. 28, 2012 having Attorney Docket No. ALI-013CIP1; U.S. patent application Ser. No. 13/433,208, filed Mar. 28, 2012 having Attorney Docket No. ALI-013CIP2; U.S. patent application Ser. No. 13/433,208, filed Mar. 28, 2012 having Attorney Docket No. ALI-013CIP3; U.S. patent application Ser. No. 13/454,040, filed Apr. 23, 2012 having Attorney Docket No. ALI-013CIP1CIP1; U.S. patent application Ser. No. 13/627,997, filed Sep. 26, 2012 having Attorney Docket No. ALI-100; all of which are incorporated herein by reference for all purposes.

In some embodiments, stressor analyzer 2224 is configured to receive activity-related data 2114 to determine stress scores that weigh against a positive affective state in favor of a negative affective state. For example, if activity-related data 2114 indicates user 402 has had little sleep, is hungry, and has just traveled a great distance, then user 2202 is predisposed to being irritable or in a negative frame of mine (and thus in a relatively “bad” mood). Also, user 2202 may be predisposed to react negatively to stimuli, especially unwanted or undesired stimuli that can be perceived as stress. Therefore, such activity-related data 2114 can be used to determine whether an intensity derived from physiological state analyzer 2222 is either negative or positive, as shown.

Emotive formation module 2223 is configured to receive data from physiological state analyzer 2222 and stressor analyzer 2224 to predict an emotion in which user 2202 is experiencing (e.g., as a positive or negative affective state). Affective state manager 2220 can transmit affective state data 2230 via network(s) to a third-party, another person (or a computing device thereof), or any other entity, as emotive feedback. Note that in some embodiments, physiological state analyzer 2222 is sufficient to determine affective state data 2230. In other embodiments, stressor analyzer 2224 is sufficient to determine affective state data 2230. In various embodiments, physiological state analyzer 2222 and stressor analyzer 2224 can be used in combination or with other data or functionalities to determine affective state data 2230.

As shown, aggregated sensor-derived values 2290 can be generated by a physiological state analyzer 2222 indicating a level of intensity. Stressor analyzer 2224 is configured to determine whether the level of intensity is within a range of negative affectivity or is within a range of positive affectivity. For example, an intensity 2240 in a range of negative affectivity can represent an emotional state similar to, or approximating, distress, whereas intensity 2242 in a range of positive affectivity can represent an emotional state similar to, or approximating, happiness. As another example, an intensity 2244 in a range of negative affectivity can represent an emotional state similar to, or approximating, depression/sadness, whereas intensity 2246 in a range of positive affectivity can represent an emotional state similar to, or approximating, relaxation. As shown, intensities 2240 and 2242 are greater than that of intensities 2244 and 2246. Emotive formulation module 2223 is configured to transmit this information as affective state data 230 describing a predicted emotion of a user. An example of affective state manager 2220 is described as a affective state prediction unit of U.S. Provisional Patent Application No. 61/705,598 filed on Sep. 25, 2012, which is incorporated by reference herein for all purposes.

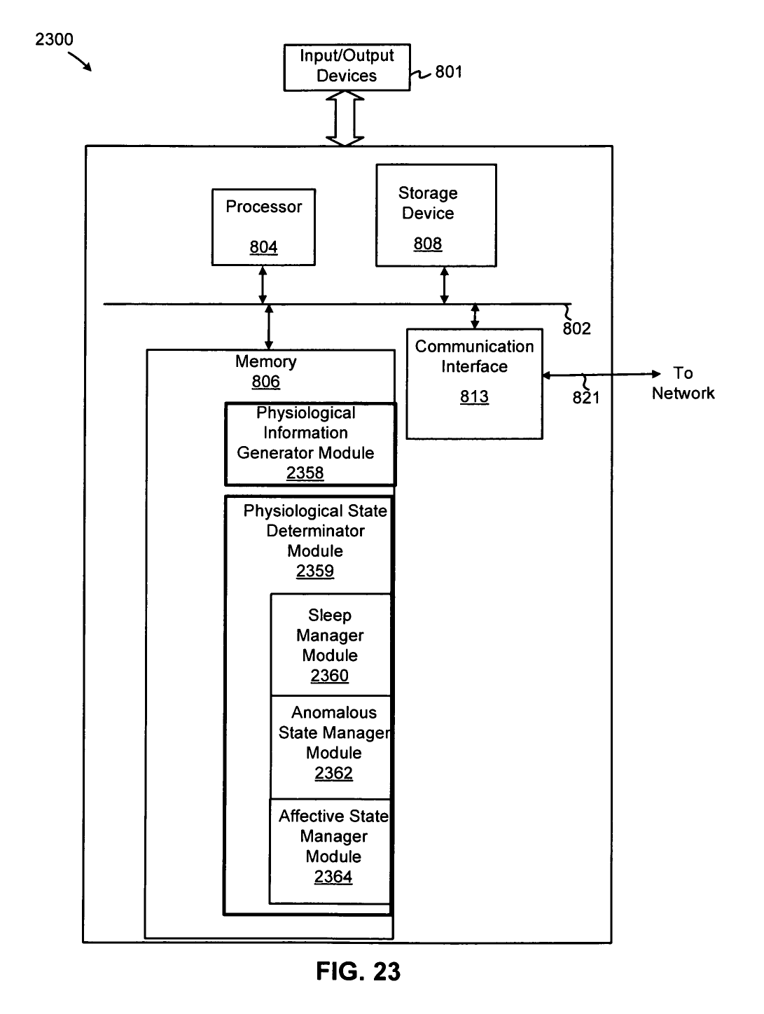

FIG. 23 illustrates an exemplary computing platform disposed in a wearable device in accordance with various embodiments. In some examples, computing platform 2300 may be used to implement computer programs, applications, methods, processes, algorithms, or other software to perform the above-described techniques, and can include similar structures and/or functions as set forth in FIG. 8. But in the example shown, system memory 806 can include various modules that include executable instructions to implement functionalities described herein. In the example shown, system memory 806 includes a physiological information generator 2358 configured to determine physiological information relating to a user that is wearing a wearable device, and a physiological state determinator 2359. Physiological state determinator 2359 can include a sleep manager module 2360, anomalous state manager module 2362, and an affective state manager module 2364, any of which can be configured to provide one or more functions described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. According to some embodiments, the term “module” can refer, for example, to an algorithm or a portion thereof, and/or logic implemented in either hardware circuitry or software, or a combination thereof. These can be varied and are not limited to the examples or descriptions provided.

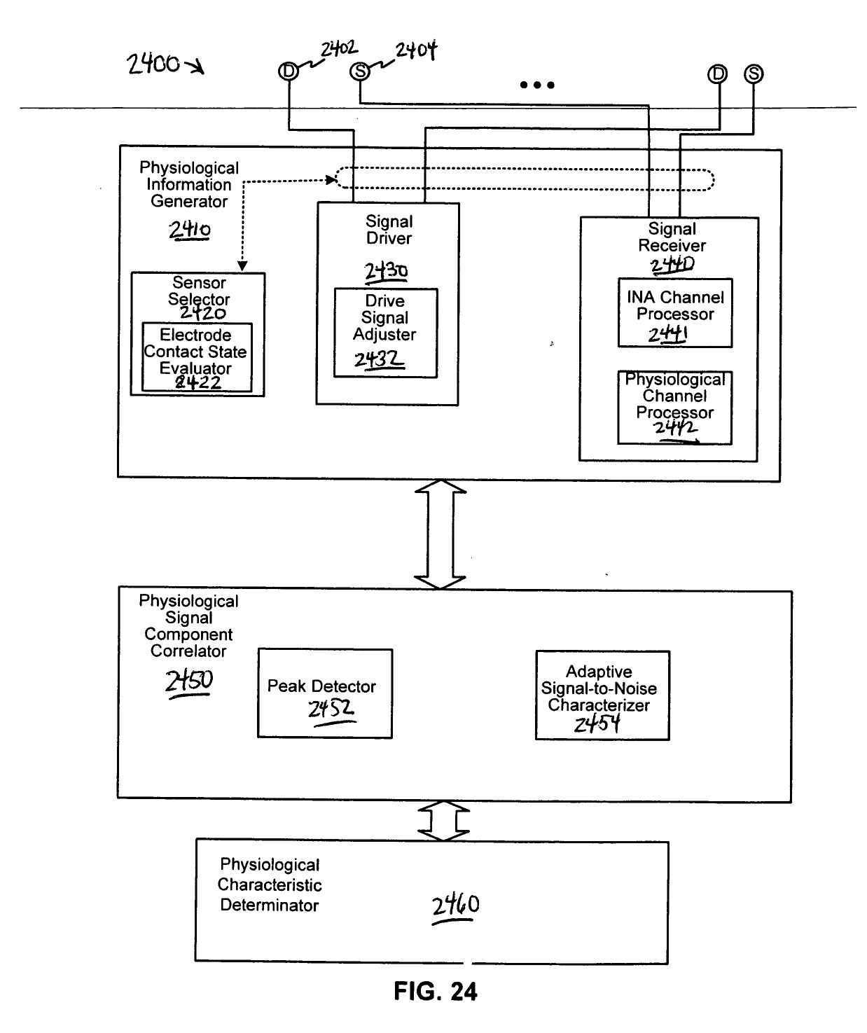

FIG. 24 is a diagram depicting examples of various components configured to determine one or more physiological characteristics, according to some examples. Diagram 2400 includes a physiological information generator 2410 including a sensor selector 2420, a signal driver 2430, and a signal receiver 2440. Physiological information generator 2410 is configured to generate data (e.g., sensor signal data) representing one or more physiological characteristics associated with a user that is wearing or carrying electrodes 2402 and 2404. Diagram 2400 also depicts a physiological signal component correlator 2450 including a peak detector 2452 and an adaptive signal-to-noise (“SNR”) characterizer 2454, and a physiological characteristic determinator 2460. In some examples, the components depicted in diagram 2400 may be disposed in a wearable device including a wearable housing and any number of electrodes or any number of the electrode pairs. An array of electrodes may be disposed at a surface of the wearable housing (e.g., a surface confronting tissue of a user), at least a portion of the array including a first subset of electrodes 2402 configured to drive a first signal to a target location and a second subset of electrodes 2404 configured to receive a second signal from the target location. The second signal may be a sensor signal that includes physiological signal components from which data representing one or more physiological characteristics may be extracted.

Signal driver 2430 may include a drive signal adjuster 2432, according to some examples. Drive signal adjuster 2432 may be configured to determine a drive signal magnitude (e.g., a drive current magnitude) for a bioimpedance signal to capture a sensor signal that includes data representing a physiological-related component (e.g., pre-processed signal data including data representing physiological characteristics, such heart rate, a galvanic skin response value (“GSR”), a respiration rate, an amount of calories expended, a rate at which energy is expended, etc. Drive signal adjuster 2432 also may be configured to select the drive current signal magnitude as a function of an impedance of a sample of a tissue (e.g., skin, vascular structures, interstitial cellular structures, etc.) such as that of a user. Signal driver 2430 is configured to drive the bioimpedance signal to one or more drive electrodes, such as electrode 2402, that are configured to convey the bioimpedance signal to a sample of tissue (e.g., at a wrist or any other portion of an organism).

In view of the foregoing, drive signal adjuster 2432 may include hardware and/or software configured to apply adjustable current signal magnitude to tissue, for example, responsive to an impedance value associated with an impedance value associated with an interface at which electrodes 2402 contact (or nearly contact) tissue. In some instances, an inherent impedance value associated with the interface between the electrode and tissue may arise due to an electrochemical interaction (e.g., between electrons from a current signal and ions associated with biological tissue). As this biologically-induced impedance may vary from person to person (e.g., to different levels of hydration, different minerals and/or nutrients consumed, etc.), drive signal adjuster 2432 is configured to adjust a drive current magnitude to accommodate various values of impedance at the electrode-tissue interface. As an example, drive signal adjuster 2432 may be configured to adjust a drive current responsive to changes in impedance caused by a buildup of sweat, a movement to other adjacent tissue, etc. Further, drive signal adjuster 2432 may be configured to determine a dynamic range of operation based on the impedance of the sample of tissue, and to select a drive current magnitude for the dynamic range of operation.

Sensor selector 2420 includes an electrode contact state evaluator 2422, which is configured to determine a state of contact for one or more drive electrodes 2402 and one or more sink electrodes 2404 (or pick-up electrodes 2404). In some examples, electrode contact state evaluator 2422 is configured to determine whether an electrode is contacting (or is sufficiently contacting) tissue. To illustrate, consider a case in which there are four electrodes composed of two pairs of drive and pick up electrodes (e.g., a tetrapolar electrode system), whereby one drive electrode 2402 is floating or otherwise not in contact with tissue. Electrode contact state evaluator 2422 can detect the now “tripolar” electrode system, and can generate data indicating such state. Other components of physiological information generator 2410 may use this information, such as drive signal adjuster 2432 to determine or select a modified current profile or magnitude with which to apply to the drive electrode in contact with tissue.

View of the foregoing, electrode contact data evaluator 2422 facilitates physiological characteristics determination in cases in which less than all electrodes are in contact with tissue. Further, electrode contact state evaluator 2422 can determine a state in which a negligible amount (e.g., none) of the electrodes are in contact with tissue, and then can generate data indicating that a wearable device including electrodes 2402 and 2404 are “off body.” Thus, bioimpedance drive signals may cease or otherwise be reduced and frequency so as to save or otherwise conserve power.

Signal receiver 2440 includes one or more channel processors configured to process (e.g., amplify and/or filter) and one or more signal channels, and is configured to receive sensor data signals from one or more sink electrodes 2404, the sensor data signals including received bioimpedance signals that include a physiological signal components. In the example shown, signal receiver 2440 includes one or more first gain amplifiers, such as an instrumentation amplifier (“INA”) channel processor 2441, and one or more second gain amplifiers, such as a physiological channel processor 2442. According to some examples, one or more physiological channel processors 2442 can adjust and apply gain configured for a specific physiological characteristic, such as heart rate (e.g., heart rate channel), respiration rate (e.g., respiration rate channel), and/or galvanic skin response (“GSR”) (galvanic skin response channel), etc.

In view of the foregoing, one or more gain amplifiers of a signal receiver 2440 may be configured to adjust gain based on an impedance of the tissue and/or body of an organism, according to some embodiments. A gain for instrumentation amplifier (“INA”) channel processor 2441 can be adjusted based on, for example, a received bioimpedance signal or sensors data signal, which, in turn, may be derived from an adjusted drive current. With adjustable drive currents and adjustable gains for each channel, signal receiver 2440 may facilitate an optimized or otherwise enhanced signal-to-ratio (“SNR”).

Physiological signal component correlator 2450 is configured to extract one or more physiological characteristics from a portion of a physiological-related signal component. According to some embodiments, physiological signal component correlator 2450 and/or one or more of its components (e.g., some or all components) may be configured to operate in a time domain rather than in a frequency domain. As shown, physiological signal component correlator 2450 may include a peak detector 2452 and an adaptive signal-to-noise characterizer 2454. Peak detector 2452 is configured to detect a portion of the physiological-related signal component for determining whether the portion includes data representative of a physiological characteristic, such as data indicative of a heart rate or pulse wave. Peak detector 2452 is thus configured to identify a magnitude of a sample, for example, in a window of time that may include biometric data. Adaptive signal-to-noise characterizer 2454 may be configured to determine data representing a value of a signal-to-noise ratio for a portion of a received bioimpedance signal (e.g., a second signal, such as an amplified signal from signal receiver 2440) including data representing the one or more physiological characteristics. Also, adaptive signal-to-noise characterizer 2454 may be configured to adapt the value of a signal-to-noise ratio over time and/or for other samples. Note that adaptive signal-to-noise characterizer 2454 may be configured to adapt the value of a signal-to-noise ratio as a function of an impedance of tissue. Note, in accordance with some embodiments, physiological signal component correlator 2450 may include one or more components bid to operate in the frequency domain. Physiological characteristic determinator 2460 may be configured to derive (e.g., from data generated by physiological signal component correlator 2450) physiological signals representative of one or more physiological characteristics.

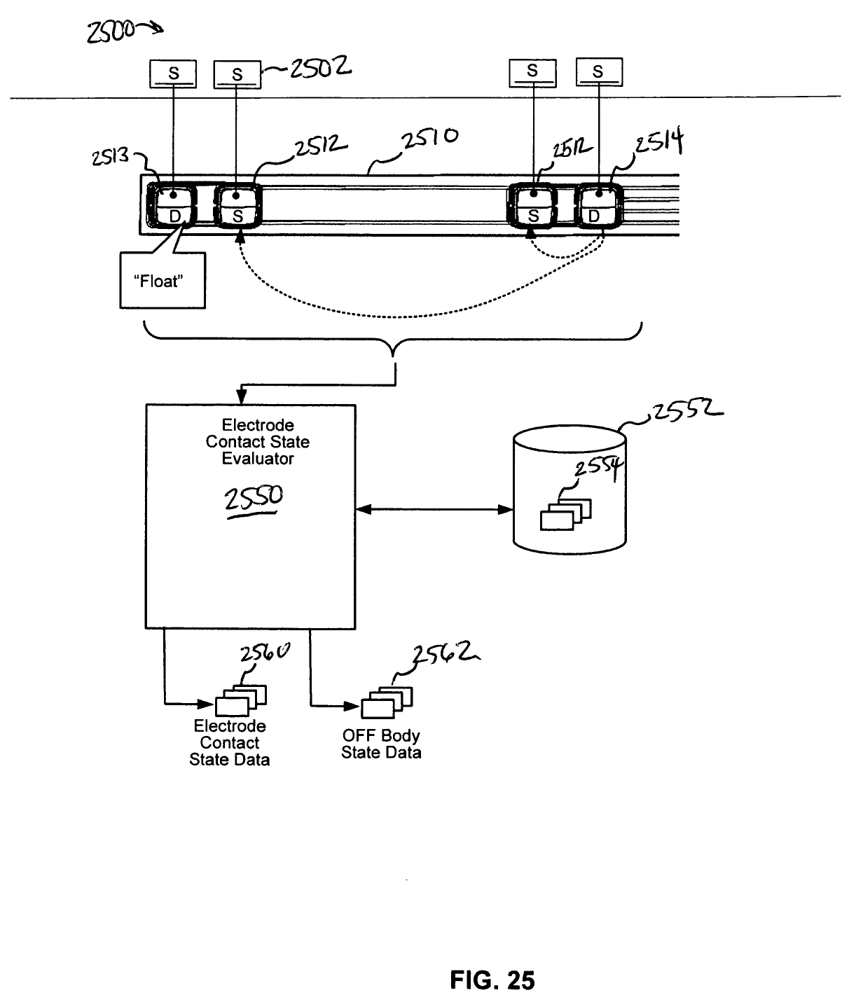

FIG. 25 is a diagram of an example of an electrode contact state evaluator, according to some examples. Diagram 2500 depicts an electrode contact state evaluator 2550 coupled to a repository 2552 including state data 2554, which includes data to determine a state for one or more electrodes and control data for transmitting to other components to, for example, generate bioimpedance signals based on state data 2554. Further, electrode contact state evaluator 2550 is configured to sense a state of contact relative to one or more electrodes 2512 in, for example, a strap 2510 or band of a wearable computing device.

In the example shown, electrode contact state evaluator 2550 can detect that drive electrode 2513 is floating or otherwise is not contacting tissue. In some cases, a bioimpedance signal may still be applied to tissue to determine physiological characteristics. For example, drive electrode 2514 can be configured to transmit a bioimpedance signal via tissue (not shown) to sink electrodes 2512. As such, electrode contact state evaluator 2550 can detect the states of each of the electrodes and retrieve control data from state data 2554, whereby the state data can be transmitted as data 2560 for other components to operate responsive to the state described above. Thus, a driver current signal magnitude may be selected on a drive current profile associated with the state of contact of for electrodes shown in diagram 2500.

In another example, electrode contact state evaluator 2550 can determine that a predominant amount of electrodes are in a state indicative of other than contacting tissue (e.g., adjacent a medium or any other material other than tissue). In this example, electrode contact state evaluator 2550 can generate “off body” state data 2562 indicating the wearable computing device is likely not worn. Further, electrodes 2512, 2513, and 2514 may be coupled such that if not coupled to tissue, electrodes may be associated with a particular state, voltage, or current (e.g., pulled up to a potential). In some examples, a DC offset may be applied to a drive electrode to facilitate ion-electron exchange

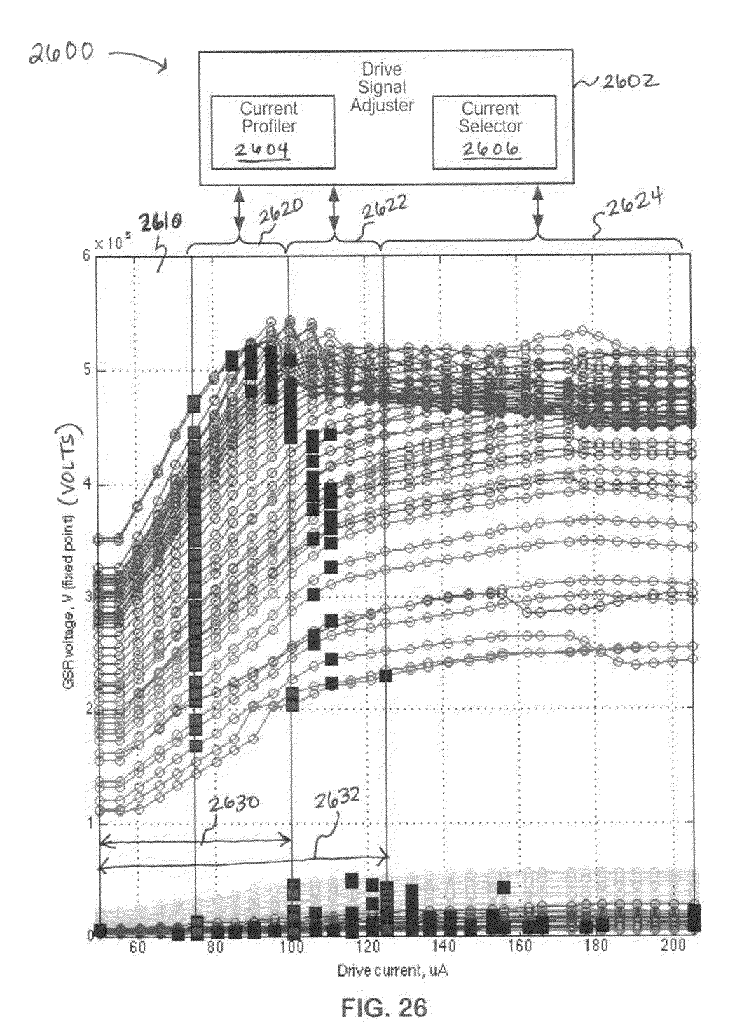

FIG. 26 is an example of a number of current profiles with which a drive signal adjuster may adjust current, according to some examples. Diagram 2600 depicts a drive signal adjuster 2602 including a current profiler 2604 and a current selector 2606.

Current profiler 2604 can be configured to profile an impedance range of a body or a tissue based on a drive current (e.g., 75 uA, 100 uA, or 120 uA). As shown, current profiler 2604 can determine a number of relationships 2610 in which a voltage between drive contacts may start to saturate. As different individuals have different chemical and biological compositions, a tissue or body impedance may vary over different drive currents. In this example, current profiler 2604 generates a number of relationships 2610 to identify three levels of impedance. A low level of impedance is related to those individuals in which saturation begins between 75 and 100 uA, a medium level of impedance is related to those individuals which saturation begins between 100 and 125 uA, and a high level of impedance is related to those individuals in which saturation occurs above 125 uA. Note that fewer or more levels of impedance can be determined for any current profile. Accordingly, different dynamic ranges 2630 and 2632 also may be determined and employed by current selector 2606 when selecting a drive current for a bioimpedance signal. In some examples, drive signal adjuster 2606 may access current profiles 2610 to drive a specific adjusted current into an electrode. Note that as an impedance of a tissue or a portion of the body changes over time, current profiler 2604 can detect such changes in apply a different current profile (e.g., a different level of impedance) when generating a bioimpedance signal.

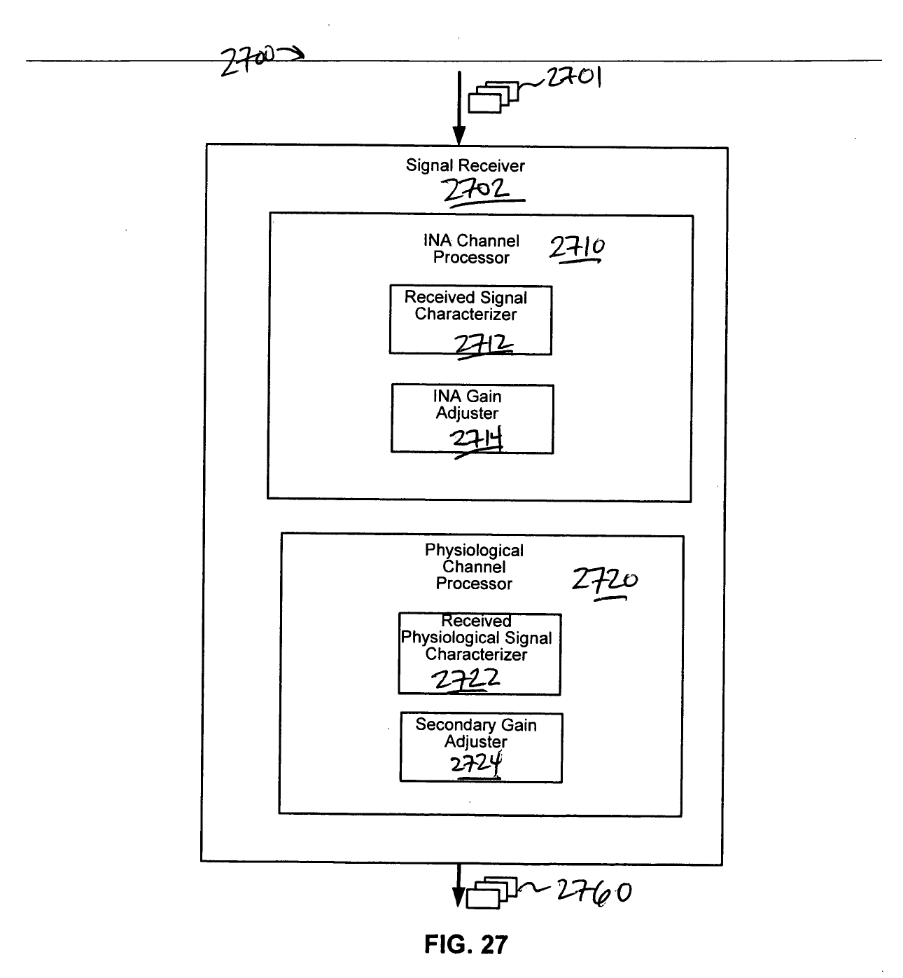

FIG. 27 is a diagram depicting a signal receiver, according to some examples. Diagram 2700 includes a signal receiver 2702 configured to receive a bioimpedance signal from an electrode, and is further configured to apply an adjusted gain to a sensor signal (or received bioimpedance signal) for generating and amplified signal 2760. Amplified signal 2760 includes data representing a portion of a physiological-related signal component that includes data representing a physiological characteristic. Signal receiver 2702 includes channel processors configured to adjust, for example, a first gain to calibrate a magnitude of a sensor signal into a range of magnitude values to form a calibrated sensor signal. The magnitude of the sensor signal may be a function of the impedance of the tissue. Further, another channel processor may be configured to adjust a second gain to calibrate a magnitude of a physiological signal into a range of physiological signal magnitude values.

Signal receiver 2702 is shown to include an instrumentation amplifier (“INA”) channel processor 2710, which, in turn, includes a received signal characterizer 2712 and a gain adjuster 2714. In one example, instrumentation amplifier (“INA”) channel processor 2710 is configured to receive a received bioimpedance signal that is composed of a carrier wave (e.g., 32 kHz square or sinusoidal waveform) and physiological-related signal components. In this example, a wearable device is configured to drive (or clock) one or more processing units (e.g., CPUs or micro controllers) at a clock rate of 32 kHz and a signal driver, according to some examples, can generate a bioimpedance signal of the same (or similar frequency) so as to minimize or otherwise reduce noise. Received signal characterizer 2712 is configured to detect a peak value over a unit of time, whereby the peak value is used to characterize an aspect of the bioimpedance signal. For example, in the 3 V powered system, a gain is selected such that an amplified signal is between 1.4 V and 1.6 V. The peak value is associated with 1.3 V or 1.7 V, INA gain adjuster 2714 is configured to adjust the gain accordingly.

Signal receiver 2702 is also shown to include a physiological channel processor 2720 that further includes a received physiological signal characterizer 2722 and a secondary gain adjuster 2724. Physiological channel processor 2720 is configured to amplify the signal associated with a physiological characteristic, such as heart rate or respiration rate. For example, consider that received physiological signal characterizer 2722 is configured to determine (e.g., digitized) a peak value, a median (or average) value, and a low value. Secondary gain adjuster 2724 is configured to use one or more thresholds for adjusting a gain of physiological channel processor 2720 so as to ensure optimal amplification for further processing.

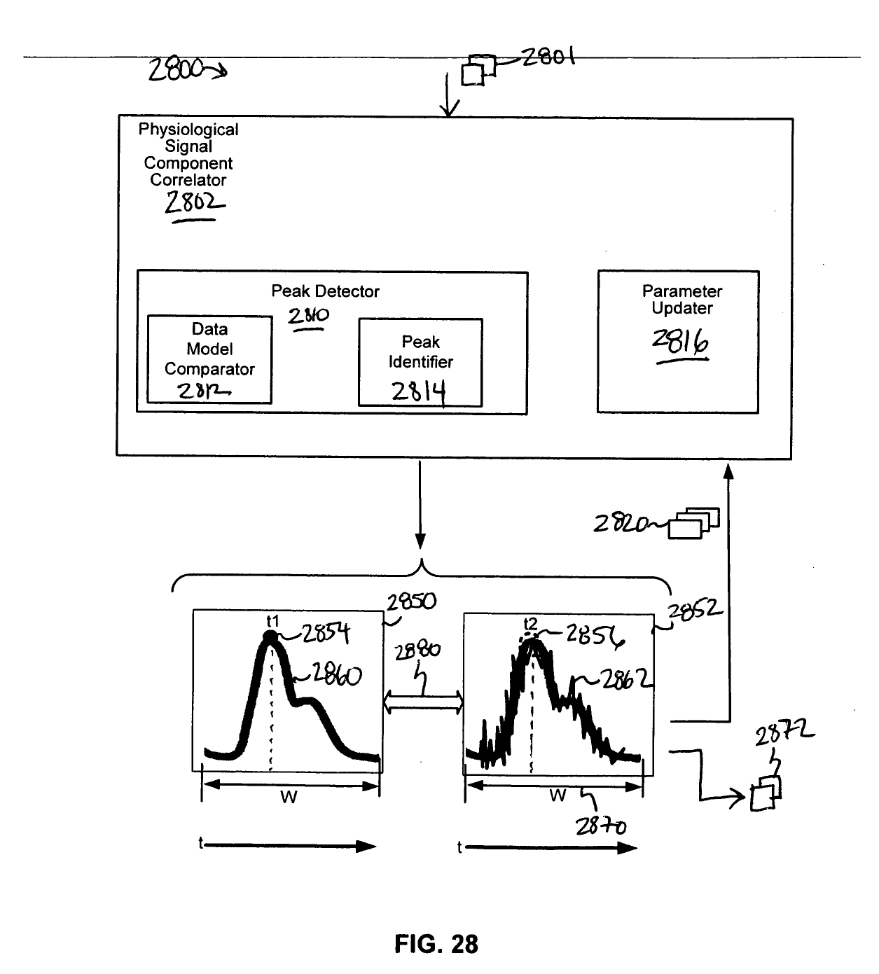

FIG. 28 is a diagram depicting a physiological signal component correlator, according to some examples. Diagram 2800 includes a physiological signal component correlator 2802 is configured to receive amplified signal data 2801, for example, from one or more above-identified amplified signal channels. Amplified signal data 2801 may include a portion of the physiological-related signal component that includes data representing a physiological characteristic, the amplified signal being derived from bioimpedance signal based on an impedance value of a tissue. As shown, physiological signal component correlator 2802 includes a peak detector 2810 and a parameter updater 2816. Peak detector 2810 is configured to determine a comparable or equivalent portion of the sample or portion of a physiological-related signal component being analyzed to contact the physiological characteristic, such as the characteristics of a pulse wave. Further, peak detector 2810 may operate, going to some examples, in the time domain to determine an equivalent portion in sampled signal (e.g., an equivalent peak magnitude in equivalent part of a sampled signal). According to some examples, operation in a time domain may employ fewer computational resources and/or power than otherwise might be the case. Thus, peak detector 2810 can operate continuous or nearly continuous mode of string processing.

To illustrate operation of peak detector 2810, consider the following. Peak detector 2810 includes a data model comparator 2812 is configured to compare a data model 2850 to a sample 2852 of amplified signal 2801 over window of time 2870. We detecting heart rate, a window 2870 may be up to, for example, 1.5 times a period between heart beats. As shown, data model 2850 is a representation of a computed or determined physiological characteristic (e.g., a pulse wave 2860 having a magnitude 2854 at time t1) based on physiological-related signal components. As shown, sample 2862 may include any number of noise components. Peak identifier 2814 may be configured to identify a magnitude 2856 (at time t2) of portion 2852 of the physiological-related signal component. Further, data model comparator 2812 may be configured to detect a match between magnitudes 2854 and 2856 (e.g., one or more peak or maximum values) to establish a matched value of a physiological characteristic, whereby the magnitudes may or may not occur substantially at the same time during a window of time, such as window of time 2870. The matched value or other representations of the detected physiological characteristic can be generated as data 2872, which may be sent directly or indirectly to a physiological characteristic determinator.

According to some examples, data model comparator 2812 is configured to perform correlation operation 2880, such as an autocorrelation operation, on the data representing data model 2870 and data representing portion 2852 of the physiological-related signal component embodied in amplified signal 2801. In other examples, data model comparator 2812 is configured to use any known techniques to determine a correlation (e.g., “sliding correlation”) between the 2850 and sample 2852. In one case, data model comparator 2812 may perform a Pearson correlation operation or the like. By correlating magnitudes and/or peak values between the data model 2850 and sample 2852, peak detector 2810 may operate insensitive or nearly insensitive to a number of maximum values or magnitudes in a signal pattern.

According to some embodiments, data model 2850 is updated periodically or a periodically to reflect a trend in changes in physiological characteristics (e.g., an increase or decrease in heart rate, respiration rate, or GSR values). Therefore, a magnitude 2856 and other characteristics of sample 2852 may be applied to adjust data model 2850. In other examples, data model 2850 may be developed and maintained as an empirically-generated signal model that includes one or more characteristics (e.g., in terms of magnitude, time, etc.) based on success of or any subsequent sample so that data model 2850 in accordance with trends in a physiological characteristic. In other examples, data model 2850 may be formed by accumulation (e.g., accumulating a signal in a window).

Data 2820 may be generated as a result of a correlation between data model 2850 and sample 2852 for subsequent processing. Data 2820 may include the value representing a peak period, a window size, a magnitude of the peak value (e.g., a time t2), and the like. Parameter updater 2816 is configured to use this or other data to update the parameters for performing peak detection. For example, data 2820 may include updated magnitudes and signal shapes for modifying the data representing data model 2850. According to some examples, physiological signal component correlator 2802 may include one or more input filters tuned for respiration rate bandwidth, heart rate bandwidth, GSR bandwidth, and the like. Note further that side peaks may correlate at least by factor 0.3 for respiration rates and 0.5 for heart rate signal components, according to some examples.

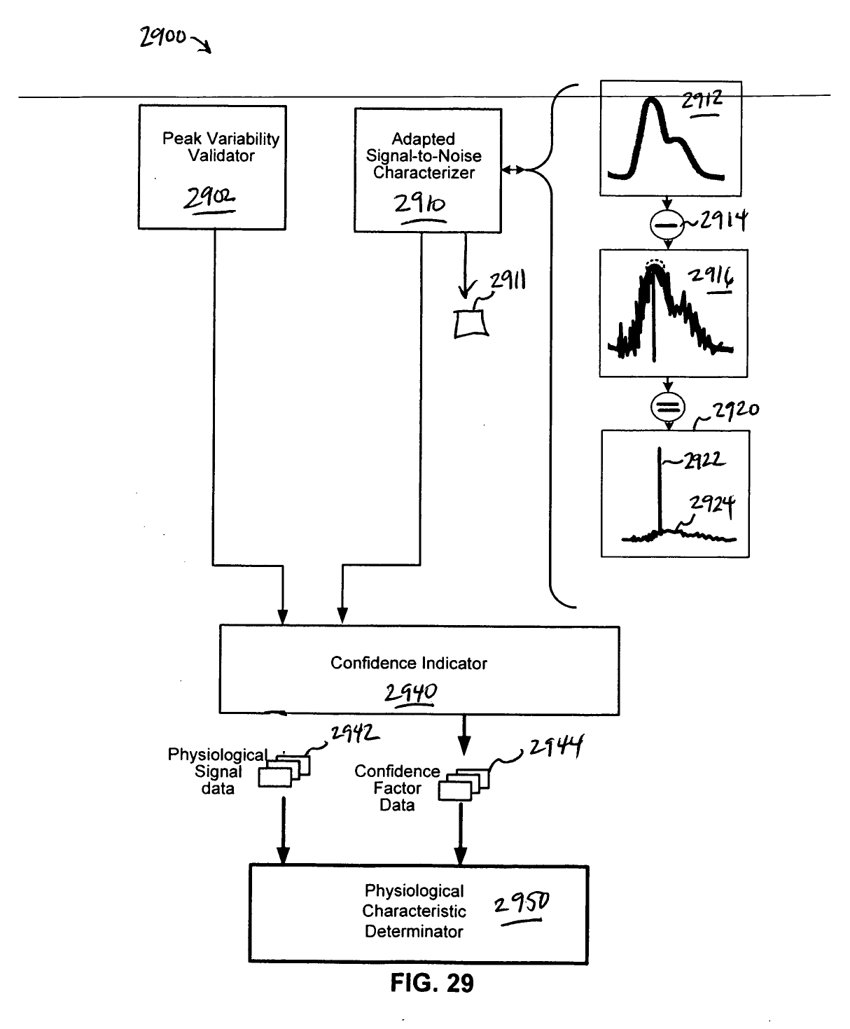

FIG. 29 is a diagram depicting an example of components of a physiological signal component correlator, according to some embodiments. Diagram 2900 includes a peak variability validator 2902, an adapted signal-to-noise characterizer 2910, and a confidence indicator generator 2940. Confidence indicator generator 2940 is configured to generate confidence factor data 2944 that is indicative of whether physiological signal data 2942 represents a valid physiological characteristic, such as a valid pulse wave magnitude. Confidence indicator generator 2940 may be configured to determine a confidence indicator value representative of a degree of likelihood that the matched value is representative of the physiological characteristic. Confidence indicator generator 2940 generates confidence factor data 2944 based on data from peak variability validator 2902 and data from adapted signal-to-noise characterizer 2910.

Peak variability validator 2902 may be configured to determining a time interval between a first magnitude (e.g., a first peak value) and another magnitude (e.g., a second peak value), and to calculate a rate indicative of the value of the physiological characteristic (e.g., a heart rate). Further, confidence indicator generator 2940 may be configured to determine a confidence indicator value based on validating that the rate is within a range of valid rates associated with the physiological characteristic. For example, confidence indicator generator 2940 may be configured to determine a validate a rate over a time interval is within a range of valid heart rates are associated with a heart rate as the physiological characteristic. In some examples, peak variability validator 2902 is configured to define or otherwise maintain time range during which a next magnitude of the physiological characteristic is expected. For example, at a heart rate of 60 bpm a next heartbeat is expected to fall at about 1 second from a previous heart beat. As such, peak variability validator 2902 detects whether a subsequent magnitude or peak value of the physiological characteristic falls outside expected changes in values of heart rates, respiration rates, GSR values, etc. In some examples, peak variability validator 2902 generates data representing either an enable signal or disable signal for generating confidence factor data 2944 by confidence indicator generator 2940.

Adapted signal-to-noise characterizer 2910 may be configured to adapt a value of a signal-to-noise ratio to form an adapted value of the signal-to-noise ratio based on a bioimpedance signal (e.g., signal based on an impedance value of tissue). Further, adapted signal-to-noise characterizer 2910 may be configured to characterize an adapted value of the signal-to-noise ratio to form a characterized value of the signal-to-noise ratio. Some cases, confidence indicator 2940 is configured to validate the characterized value of the signal-to-noise ratio for determine confidence factor data 2944. According to some embodiments, adapted signal-to-noise characterizer 2910 is configured to characterize the signal-to-noise ratio relative to the amount of noise in sample 2916 over data model 2912. Also, adapted signal-to-noise characterizer 2910 is configured to determine a signal-to-noise value that, for example may be adapted over time or may be adapted responsive to signals derived by different values of impedance (including different drive magnitudes), or both. As shown, and operation 2914 may yield a difference signal 2924 for result 2920, which indicates an amount of noise (e.g., the difference signal 2924) relative to a magnitude 2922. In some examples, the characterization value can be assigned or otherwise indicate relative value of a signal to noise ratio whereby relatively high signal-to-noise ratios may have relatively high characterization values. As such, relatively high characterization values indicate a favorable degree of quality of a signal embodying physiological characteristic. In some cases, confidence indicator generator 2940 can use any combination of the characterization value from adapted signal-to-noise characterizer 2910 and data from peak variability validator 2902 to generate confidence factor data 2944. According to some examples, the signal-to-noise ratio can be adapted or otherwise updated subsequently.

In some embodiments, physiological characteristic determinator 2950 receives confidence factor data 2944 and physiological signal data 2942 and generates the physiological signal such as a heart rate, respiration rate, and the like. Note that physiological characteristic determinator 2950 can use the confidence factor data 2944 and a variety of ways. For example, when calculating heart rate physiological characteristic determinator 2950 may receive 8 peak values and 8 corresponding confidence indicator values. Should all the confidence indicator values indicate valid data (e.g., a relatively high likelihood that the detected 8 signals and peak values are accurate), then a heart rate will be validate. But if less than 8 confidence indicator values are indicative of a relatively high likelihood of a detected heart rate, then physiological characteristic determinator 2950 may invalidate the 8 peak values or require further additional information to ensure accurate processing. Physiological characteristic determinator 2950 can determine physiological characteristic, such as heart rate, a respiration rate, a galvanic skin resistance value, data representing an affective state or mode, an amount of energy expenditure, an amount of calories expended, and the like

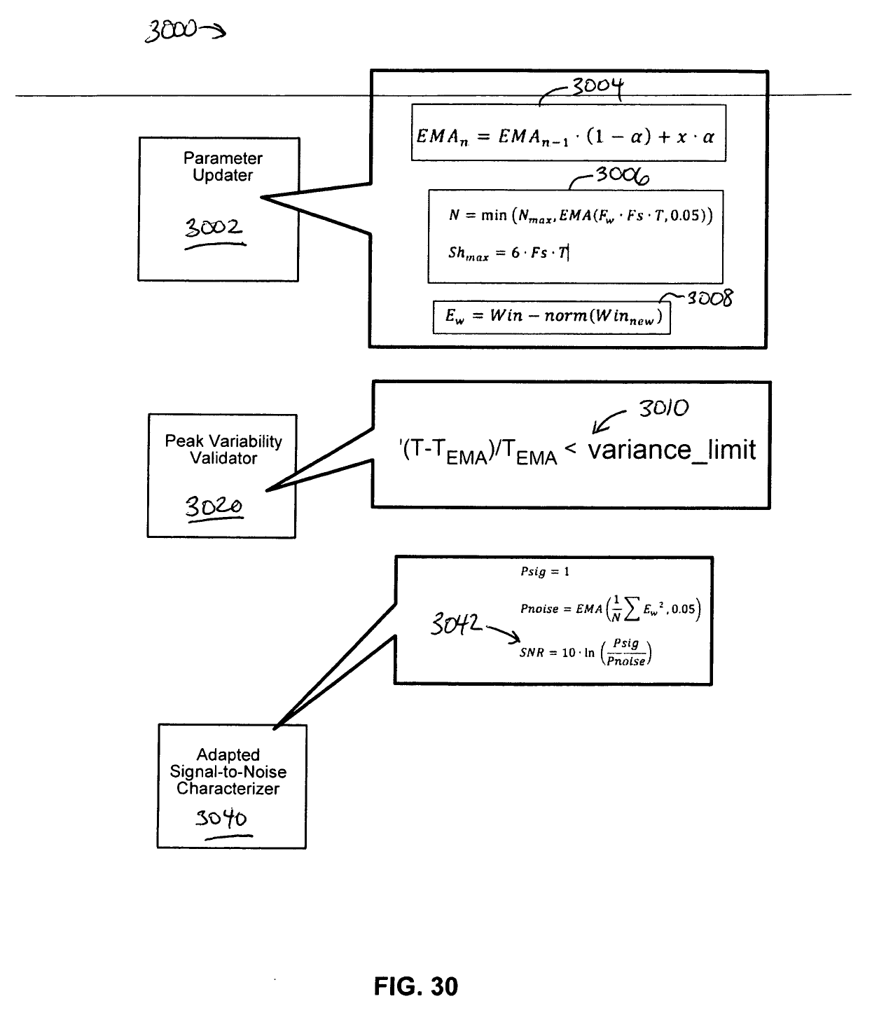

FIG. 30 is a diagram depicting exemplary operation of a parameter updater, peak variability validator, and an adapted signal-to-noise ratio characterizer, according to various examples. Diagram 3000 includes a parameter updater 3002, a peak variability validator 3020, and an adapted signal-to-noise characterizer 3040.

According to some embodiments, parameter updater 3002 may use a relationship defined by an exponential moving average (“EMA”) 3004 to estimate confidence factor (e.g., a window confidence) by applying EMA 3004 on a detected peak value. “EMAn−1” is a previous EMA value, “alpha” is a value based on a SNR value (e.g., a factor, a multiple/inverse multiple of SNR, or a speed parameter), and “x” is a new value for EMA. Also, parameter updater 3002 may update or estimate a new window size and/or a window shift at 3006 based on a new peak period, where “N” is a window size, “Fw” is a window factor (e.g., a constant value), “Nmax” is a maximum window length, “Fs” is a sample rate, “T” is a peak period, and “Shmax” represents a maximum window shift. Parameter updater 3002 may update a window error at 3008 between the latest window and a window accumulator (e.g., including a data model signal) for determining an adaptive signal-to-noise ratio.

A peak variability validator 3020 a variance at 3010 is determine to indicate whether a signal magnitude is within limits (e.g., are valid), according to some examples. In the example shown, a variance is calculated to determine whether it's below a variance limit, where T is a detected period, “TEMA” is an exponential averaging for T, and a variance_limit may be defined as a limit value (e.g., 0.2 for heart rate, or 0.4 for breathing).

Adapted signal-to noise characterizer 3040 may generate a signal to noise ratio or were a value indicative thereof. At 3042, signal-to-noise ratio is determined, where Psig represents signal power equal 1, whereby noise power is normalized to 1. Pnoise represents normalized noise power. In some examples, a value of alpha may be calculated as one tenth of a value of SNR for determining a new value of alpha that is indicative of the quality of the SNR value.

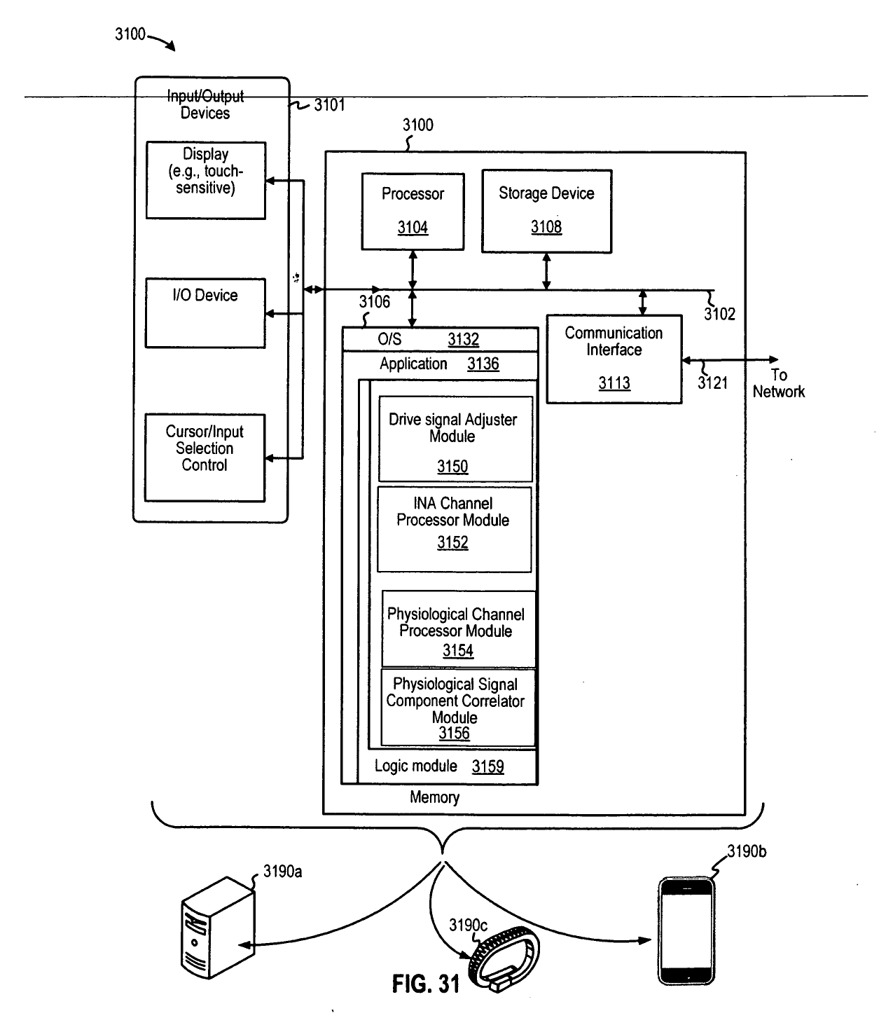

FIG. 31 illustrates an exemplary computing platform disposed in a device configured to facilitate physiological signal determination in accordance with various embodiments. In some examples, computing platform 3100 may be used to implement computer programs, applications, methods, processes, algorithms, or other software to perform the above-described techniques.

In some cases, computing platform can be disposed in wearable device 3190c or implement, a mobile computing device 3190b, or any other device, such as a computing device 3190a.

Computing platform 3100 includes a bus 3102 or other communication mechanism for communicating information, which interconnects subsystems and devices, such as processor 3104, system memory 3106 (e.g., RAM, etc.), storage device 3108 (e.g., ROM, etc.), a communication interface 3113 (e.g., an Ethernet or wireless controller, a Bluetooth controller, etc.) to facilitate communications via a port on communication link 3121 to communicate, for example, with a computing device, including mobile computing and/or communication devices with processors. Processor 3104 can be implemented with one or more central processing units (“CPUs”), such as those manufactured by Intel® Corporation, or one or more virtual processors, as well as any combination of CPUs and virtual processors. Computing platform 3100 exchanges data representing inputs and outputs via input-and-output devices 3101, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 3100 performs specific operations by processor 3104 executing one or more sequences of one or more instructions stored in system memory 3106, and computing platform 3100 can be implemented in a client-server arrangement, peer-to-peer arrangement, or as any mobile computing device, including smart phones and the like. Such instructions or data may be read into system memory 3106 from another computer readable medium, such as storage device 3108. In some examples, hard-wired circuitry may be used in place of or in combination with software instructions for implementation. Instructions may be embedded in software or firmware. The term “computer readable medium” refers to any tangible medium that participates in providing instructions to processor 3104 for execution. Such a medium may take many forms, including but not limited to, non-volatile media and volatile media. Non-volatile media includes, for example, optical or magnetic disks and the like. Volatile media includes dynamic memory, such as system memory 3106.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 3102 for transmitting a computer data signal.

In some examples, execution of the sequences of instructions may be performed by computing platform 3100. According to some examples, computing platform 3100 can be coupled by communication link 3121 (e.g., a wired network, such as LAN, PSTN, or any wireless network, including WiFi of various standards and protocols, Blue Tooth®, Zig-Bee, etc.) to any other processor to perform the sequence of instructions in coordination with (or asynchronous to) one another. Computing platform 3100 may transmit and receive messages, data, and instructions, including program code (e.g., application code) through communication link 3121 and communication interface 3113. Received program code may be executed by processor 3104 as it is received, and/or stored in memory 3106 or other non-volatile storage for later execution.

In the example shown, system memory 3106 can include various modules that include executable instructions to implement functionalities described herein. System memory 3106 may include an operating system (“O/S”) 3132, as well as an application 3136 and/or logic module 3159. In the example shown, system memory 3106 includes a drive signal adjuster module 3150, INA channel processor module 3152, physiological channel processor module 3154, and physiological signal component correlator module 3156 (collectively “the Depicted Modules”) including any number of modules (not shown), one or more of which can be configured to provide or consume outputs to implement one or more functions described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. According to some embodiments, the term “module” can refer, for example, to an algorithm or a portion thereof, and/or logic implemented in either hardware circuitry or software, or a combination thereof. These can be varied and are not limited to the examples or descriptions provided.

In some embodiments, the Depicted modules or one or more of their components (e.g., a motion recovery controller), or any process or device described herein, can be in communication (e.g., wired or wirelessly) with a mobile device, such as a mobile phone or computing device, or can be disposed therein.

In some cases, a mobile device, or any networked computing device (not shown) in communication with the Depicted modules or one or more of their components (or any process or device described herein), can provide at least some of the structures and/or functions of any of the features described herein. As depicted in the above-described figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in any of the figure can represent one or more algorithms. Or, at least one of the elements can represent a portion of logic including a portion of hardware configured to provide constituent structures and/or functionalities.

For example, the Depicted modules, or any of their one or more components, or any process or device described herein, can be implemented in one or more computing devices (i.e., any mobile computing device, such as a wearable device, an audio device (such as headphones or a headset) or mobile phone, whether worn or carried) that include one or more processors configured to execute one or more algorithms in memory. Thus, at least some of the elements in the above-described figures can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit.

For example, the Depicted modules, including one or more components, or any process or device described herein, can be implemented in one or more computing devices that include one or more circuits. Thus, at least one of the elements in the above-described figures can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A method comprising:
determining a drive signal magnitude for a bioimpedance signal to capture a sensor signal including data representing a physiological-related component;
selecting the drive signal magnitude as a function of an impedance of a sample of a tissue;
driving the bioimpedance signal to one or more drive electrodes that are configured to convey the bioimpedance signal to the sample of tissue;
adapting data representing a value a signal-to-noise (“SNR”) ratio as a function of the impedance of the sample of the tissue to form an adaptive signal-to-noise value;
detecting a portion of the physiological-related signal component from a received bioimpedance signal as the sensor signal based on the adaptive signal-to-noise value; and
deriving a physiological characteristic from the physiological-related signal component.

2. The method of claim 1, wherein selecting the drive signal magnitude comprises:
selecting a drive current magnitude.

3. The method of claim 1, wherein selecting the drive signal magnitude comprises:
determining a dynamic range of operation based on the impedance of the sample of tissue; and
selecting a drive current magnitude for the dynamic range of operation.

4. The method of claim 1, further comprising:
adjusting a gain of a channel based on impedance of the sample of the tissue.

5. The method of claim 4, wherein adjusting the gain of the channel comprises:
adjusting the gain of the channel based on the received bioimpedance signal.

6. The method of claim 1, wherein detecting a portion of the physiological-related signal component comprises:
extracting the portion of the physiological-related signal component in the time-domain.

7. The method of claim 1, further comprising:
determining a state of contact for the drive electrodes and sink electrodes; and
generating data representing the state of contact for the drive electrodes and the sink electrodes.

8. The method of claim 1, wherein deriving the physiological characteristic from the physiological-related signal component comprises:
determining a heart rate (“HR”) signal, a galvanic skin response (“GSR”) signals, or a respiration rate (“RR”) signal as the physiological characteristic.

9. The method of claim 8, wherein deriving the physiological characteristic from the physiological-related signal component further comprises:
calculating a first value representing a maximal oxygen consumption (“VO2 max”) or a second value representing pulse or blood pressure based one or more of the HR and the respiration signal.

10. An apparatus comprising:
a wearable housing;
an array of electrodes disposed at a surface of the wearable housing, at least a portion of the array including electrodes configured to either drive a first signal to a target location or receive a second signal from the target location, the second signal including data representing one or more physiological characteristics;
a signal driver configure to apply an adjustable current signal to a subset of the electrodes, a magnitude of the adjustable current signal being a function of an impedance of tissue;
an adaptive signal-to-noise characterizer configured to determine data representing a value of a signal-to-noise ratio for a portion of the second signal including data representing the one or more physiological characteristics, and further configured to adapt the value of a signal-to-noise ratio; and
a physiological characteristic determinator configured to derive physiological signals representative of one or more physiological characteristics.

11. The apparatus of claim 10, wherein the one or more physiological characteristics comprise one or more of a heart rate, a respiration rate, a galvanic skin resistance value, data representing an affective state or mode, an amount of energy expenditure and an amount of calories expended.

12. The apparatus of claim 10, further comprising:
a drive signal adjuster configured to determine a drive signal magnitude for a bioimpedance signal, and further configured to select the drive signal magnitude as a function of the impedance of the tissue.

13. The apparatus of claim 10, further comprising:
an instrumentation amplifier channel processor configured to determine a first gain, and further configured to apply the first gain; and
a physiological channel processor configured to determine a second gain, and further configured to apply the second gain.

14. The apparatus of claim 13, wherein the second gain is adapted to amplify a heart rate signal or a respiration signal.
